(19) United States
(12) Patent Application Publication
(10) Pub. No.: US 2023/0147733 A1
(43) Pub. Date: May 11, 2023

(54) OXIDIZED TUMOR CELL LYSATES ENCAPSULATED IN LIPOSOMAL SPHERICAL NUCLEIC ACIDS AS POTENT CANCER IMMUNOTHERAPEUTICS

(71) Applicant: NORTHWESTERN UNIVERSITY, Evanston, IL (US)

(72) Inventors: Chad A. Mirkin, Wilmette, IL (US); Cassandra Elizabeth Callmann, Evanston, IL (US)

(21) Appl. No.: 17/917,871

(22) PCT Filed: Apr. 9, 2021
(86) PCT No.: PCT/US21/26624

§ 371 Date: Oct. 7, 2022

Related U.S. Application Data
(60) Provisional application No. 63/008,229, filed on Apr. 10, 2020.

(51) Int. Cl. A61K 39/00; C12N 5/09; C12N 15/117; A61K 47/69; A61K 47/64; A61K 47/54; A61P 35/00

(52) U.S. Cl. CPC A61K 39/0011; C12N 5/0693; C12N 15/117; A61K 47/6915; A61K 47/64; A61K 47/545; A61P 35/00; C12N 2310/17; C12N 2310/315; C12N 2310/3515

(57) ABSTRACT

The disclosure is generally related to nanoparticles have an oxidized tumor cell lysate encapsulated therein and oligonucleotides on the surface thereof. Methods of making and using the nanoparticles are also provided herein.

### CROSS-REFERENCE TO RELATED APPLICATIONS

This application claims the priority benefit under 35 U.S.C. § 119(e) of U.S. Provisional Patent Application No. 63/008,229, filed Apr. 10, 2020, which is incorporated herein by reference in its entirety.

### STATEMENT OF GOVERNMENT SUPPORT

This invention was made with government support under CA199091 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2020-042P_Seqlisting.txt”, which was created on Apr. 10, 2020 and is 1,047 bytes in size. The subject matter of the Sequence Listing is incorporated herein in its entirety by reference.

### FIELD OF THE INVENTION

The disclosure is generally related to nanoparticles having an oxidized tumor cell lysate encapsulated therein and oligonucleotides on the surface thereof. Methods of making and using the nanoparticles are also provided herein.

### BACKGROUND

Mobilizing the immune system against tumors is a central goal of personalized cancer treatments. Indeed, the identification of tumor-associated antigens (TAAs) and the advent of cell-based therapies represent significant progress towards achieving this aim (1-5). However, these approaches, including the use of dendritic cell (DC) vaccines (6) and CAR-T cell therapies (7), are expensive and labor-intensive, as they require the extraction of immature immune cells from patients, expansion of cells ex vivo, incubation with TAAs, and reinfusion to the patient. Furthermore, these therapies are restricted to the subset of patients whose tumors express known TAAs (8), and raising an immune response with single-antigen vaccines may ultimately have limited efficacy due to tumor heterogeneity and loss of antigen expression over time (9-11).

### SUMMARY

Therapies that utilize tumor associated antigens (TAAs) have been developed, but these approaches are restricted to the subset of patients whose tumors express known TAAs and raising an immune response with single-antigen vaccines may ultimately have limited efficacy due to tumor heterogeneity and loss of antigen expression over time. Utilizing lysates isolated from tumor cells as the antigen source in cancer immunotherapies addresses these concerns; however, direct vaccination using tumor lysates has been met with limited success because of their minimal immunogenicity. Oxidizing tumor cells prior to lysate isolation and preparation significantly increases immunogenicity when the lysates are utilized as the antigen source, but the underlying mechanism of how these changes facilitate antigen presentation and alter the tumor microenvironment (TME) remain unclear. Moreover, a major challenge in immunotherapeutic development is the selection of the appropriate vehicle for delivering both adjuvant and antigen, as the way that components are formulated can significantly influence delivery to the immune system and thus, activation of immunostimulatory pathway. Nanoscale therapeutics have shown promise in this regard, in enhancing antigen-presenting cell (APC) activation over mixtures of adjuvant and antigen, because codelivery of adjuvant and antigen is required for the most potent immune response. Encapsulating oxidized tumor cell lysates in the core of spherical nucleic acids (SNAs) decorated with immunostimulatory nucleic acids enables high codelivery to the same target immune cell, which leads to superior antitumor efficacy and survival, as well as a drastically altered TME, relative to SNAs containing non-oxidized lysates and mixtures of oxidized lysates with adjuvant DNA (for example and without limitation, Toll-like receptor 9 (TLR9) agonists). The present disclosure demonstrates that the way lysates are processed, packaged, and presented to the immune cell is a critical determinant of the therapeutic potential of lysate-based immunotherapeutics.

Accordingly, in some aspects the disclosure provides a nanoparticle having a substantially spherical geometry comprising an oligonucleotide conjugated thereto, wherein the oligonucleotide is a Toll-Like Receptor (TLR) agonist, and wherein an oxidized tumor cell lysate is encapsulated within the nanoparticle. In some embodiments, the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLR5) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof. In preferred embodiments, the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof. In some embodiments, the TLR9 agonist is 5′-TCCATGACGTTCCTGACGTT-3′ (SEQ ID NO: 1). In further embodiments, the nanoparticle is a poly(lactic-co-glycolic acid) (PLGA), poly(acrylate), or poly(methacrylate) nanoparticle. In some embodiments, the TLR9 agonist is 5′-TCCATGACGTTCCTGACGTT(Spacer-18 (hexaethyleneglycol))₂Cholesterol-3′ (SEQ ID NO: 2). In some embodiments, the oligonucleotide comprises a lipophilic group. In further embodiments, the lipophilic group comprises tocopherol or cholesterol. In some embodiments, the cholesterol is cholesteryl-triethyleneglycol (cholesteryl-TEG). In some embodiments, tocopherol is a tocopherol derivative, alpha-tocopherol, beta-tocopherol, gamma-tocopherol, or delta-tocopherol. In some embodiments, the nanoparticle comprises a plurality of lipid groups. In further embodiments, at least one lipid group is of the phosphatidylcholine, phosphatidylglycerol, or phosphatidylethanolamine family of lipids. In still further embodiments, at least one lipid group is 1,2-dioleoyl-sn-glycero-3-phosphocholine (DOPC), 1,2-dimyristoyl-sn-phosphatidylcholine (DMPC), 1,2-dihexadecanoyl-sn-glycero-3-phosphatidylcholine (DPPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1-oleoyl-2-hydroxy-sn-glycero-3-phosphoethanolamine, 1-stearoyl-2-hydroxy-sn-glycero-3-phosphoethanolamine, 1,2-distearoyl-sn-glycero-3-phosphoethanolamine, and 1,2-dielaidoyl-sn-glycero-3-phosphoethanolamine, 1,2-dioleoyl-sn-glycero-3-phosphoethanolamine-N-[azido(polyethylene glycol)] (DOPE-PEG-azide), 1,2-dioleoyl-sn-glycero-3-phosphoethanolamine-N-[maleimide(polyethylene glycol)] (DOPE-PEG-maleimide), 1,2-dipalmitoyl-sn-glycero-3-phosphoethanolamine-N-[azido(polyethylene glycol)] (DPPE-PEG-azide), 1,2-dipalmitoyl-sn-glycero-3-phosphoethanolamine-N-[maleimide(polyethylene glycol)] (DPPE-PEG-maleimide), 1,2-distearoyl-sn-glycero-3-phosphoethanolamine-N-[azido(polyethylene glycol)] (DSPE-PEG-azide), and 1,2-distearoyl-sn-glycero-3-phosphoethanolamine-N-[maleimide(polyethylene glycol)] (DSPE-PEG-maleimide), 1,2-dihexadecanoyl-sn-glycero-3-phosphoethanolamine (DPPE), or a combination thereof. In some embodiments, the plurality of lipid groups comprises 1,2-dipalmitoyl-sn-glycero-3-phosphocholine (DPPC). In some embodiments, the plurality of lipid groups comprises 1,2-dioleoyl-sn-glycero-3-phosphocholine (DOPC). In some embodiments, the diameter of the nanoparticle is about 20 nanometers (nm) to about 150 nm. In some embodiments, diameter of the nanoparticle is less than or equal to about 100 nanometers. In further embodiments, diameter of the nanoparticle is less than or equal to about 80 nanometers. In some embodiments, the nanoparticle comprises about 10 to about 200 oligonucleotides. In further embodiments, the nanoparticle comprises 75 oligonucleotides. In some embodiments, the ratio of oligonucleotide to tumor cell lysate is about 0.5 nmol to about 25 nmol: about 5 μg to about 150 μg. In further embodiments, the ratio of oligonucleotide to tumor cell lysate is about 5 nmol oligonucleotide:20 μg tumor cell lysate. In some embodiments, the oxidized tumor cell lysate is derived from a tumor cell exposed to hypochlorous acid (HOCl), hydrogen peroxide, sodium hypochlorite, sodium chlorite, nitric acid, or sulfur. In some embodiments, the tumor cell is exposed to about 10 μM to about 100 μM HOCl. In further embodiments, the tumor cell is exposed to about 60 μM HOCl. In some embodiments, the tumor cell lysate is derived from a breast cancer cell, peritoneum cancer cell, cervical cancer cell, colon cancer cell, rectal cancer cell, esophageal cancer cell, eye cancer cell, liver cancer cell, pancreatic cancer cell, larynx cancer cell, lung cancer cell, skin cancer cell, ovarian cancer cell, prostate cancer cell, stomach cancer cell, testicular cancer cell, thyroid cancer cell, brain cancer cell, or a combination thereof. In further embodiments, the tumor cell lysate is derived from a triple negative breast cancer (TNBC) cell. In some embodiments, the oligonucleotide is DNA. In some embodiments, the oligonucleotide is RNA. In some aspects, the disclosure provides a pharmaceutical formulation comprising a nanoparticle having a substantially spherical geometry comprising an oligonucleotide conjugated thereto, wherein the oligonucleotide is a Toll-Like Receptor (TLR) agonist, and wherein an oxidized tumor cell lysate is encapsulated within the nanoparticle, and a pharmaceutically acceptable carrier or diluent.

In some aspects, the disclosure provides a method of making a liposomal nanoparticle comprising: exposing a tumor cell to an oxidizing agent to produce an oxidized tumor cell; then isolating lysate from the oxidized tumor cell; then contacting a lipid film with the lysate to produce a small unilamellar vesicle (SUV) comprising the lysate encapsulated therein; then adding an oligonucleotide to the SUV to make the liposomal nanoparticle. In some embodiments, the oxidizing agent is hypochlorous acid (HOCl). In some embodiments, the tumor cell is exposed to about 10 μM to about 100 μM of the oxidizing agent. In further embodiments, the tumor cell is exposed to about 60 μM HOCl. In some embodiments, the oligonucleotide is a Toll-Like Receptor (TLR) agonist. In further embodiments, the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLRS) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof. In preferred embodiments, the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof. In some embodiments, the liposomal nanoparticle is about 50 nanometers (nm) to about 100 nanometers (nm) in diameter. In further embodiments, the liposomal nanoparticle is less than about 100 nm in diameter. In still further embodiments, the liposomal nanoparticle is about 80 nm in diameter. In some embodiments, the oligonucleotide is an oligonucleotide-lipid conjugate containing a lipophilic tethered group, wherein said lipophilic tethered group is adsorbed into the surface of the SUV. In some embodiments, the lipophilic tethered group comprises tocopherol or cholesterol. In further embodiments, tocopherol is a tocopherol derivative, alpha-tocopherol, beta-tocopherol, gamma-tocopherol, or delta-tocopherol. In some embodiments, the oligonucleotide comprises RNA or DNA. In some embodiments, the oligonucleotide is DNA. In some embodiments, the oligonucleotide is a modified oligonucleotide. In some embodiments, the ratio the ratio of oligonucleotide to tumor cell lysate is about 0.5 nmol to about 25 nmol: about 5 μg to about 150 μg. In further embodiments, the ratio of oligonucleotide to tumor cell lysate is about 5 nmol oligonucleotide:20 μg tumor cell lysate.

In some aspects, the disclosure provides an antigenic composition comprising a nanoparticle having a substantially spherical geometry comprising an oligonucleotide conjugated thereto, wherein the oligonucleotide is a Toll-Like Receptor (TLR) agonist, and wherein an oxidized tumor cell lysate is encapsulated within the nanoparticle, in a pharmaceutically acceptable carrier, diluent, stabilizer, preservative, or adjuvant, or a pharmaceutical formulation of the disclosure, wherein the antigenic composition is capable of generating an immune response including antibody generation or a protective immune response in a mammalian subject. In some embodiments, the antibody response is a neutralizing antibody response or a protective antibody response.

In some aspects, the disclosure provides a method of producing an immune response to cancer in a subject, comprising administering to the subject an effective amount of an antigenic composition of the disclosure, thereby producing an immune response to cancer in the subject. In some embodiments, the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof. In some embodiments, the cancer is breast cancer. In further embodiments, the breast cancer is triple negative breast cancer (TNBC).

In some aspects, the disclosure provides a method of treating cancer in a subject in need thereof, comprising administering to the subject an effective amount of a nanoparticle having a substantially spherical geometry comprising an oligonucleotide conjugated thereto, wherein the oligonucleotide is a Toll-Like Receptor (TLR) agonist, and wherein an oxidized tumor cell lysate is encapsulated within the nanoparticle; an antigenic composition; or a pharmaceutical formulation of the disclosure, thereby treating cancer in the subject. In some embodiments, the administering is subcutaneous. In further embodiments, the administering is intravenous, intraperitoneal, intranasal, or intramuscular.

Applications of the technology disclosed herein include, but are not limited to:
Cancer immunotherapy and cancer vaccines
Vaccination against a wide range of tumor associated antigens
Personalized cancer therapy
Increasing immunogenicity of protein lysates
Stimulation of antigen presenting cells
Altering the tumor microenvironment
Immunomodulation

Advantages of the technology disclosed herein include, but are not limited to:
increased immunogenicity of tumor lysates after isolation
high codelivery of adjuvant and antigen to same target immune cell in vivo, because both components are packaged together on the nanoscale
direct application of lysate-containing immunotherapeutics, without the need for ex vivo immune cell expansion and activation
protection of lysates from degradation due to their encapsulation within the nanoparticle core
altered immune cell population

### BRIEF DESCRIPTION OF THE FIGURES

FIG. 1 shows a lysate-loaded, immunostimulatory spherical nucleic acid (Lys-SNA). (a) A schematic of a Lys-SNA. TNBC lysates (orange) from either oxidized or non-oxidized TNBC cells are encapsulated in the core of a liposome (purple), which is functionalized with cholesterol-modified nucleic acids (green) to generate the SNA. (b) Cryo-TEM of Lys-SNAs. (c) Gel electrophoresis of free CpG-1826 (left lane), Lys-SNAs (middle lane), and Lys-SNAs after exposure to Triton-X to break apart liposomes (right lane). (d) Hydrodynamic diameter of lysate-loaded liposomes and SNAs, as measured by DLS.

FIG. 2 shows a representative example of CD8+ (cytotoxic) T cell population at the tumor site at day 11 post-inoculation.

FIG. 3 shows a representative example of MDSC population at the tumor site at day 11 post-inoculation.

FIG. 4 shows lysate-loaded SNA formation from lysate-loaded liposomes. CpG-1826 is 3′-modified with cholesterol to induce bilayer intercalation.

FIG. 5 shows delivery of FITC-labeled lysate within Cy5-labeled SNAs. (a) Confocal microscopy images of BMDCs incubated with dual fluorophore-labeled Lys-SNA and Lys-Mix for 1 and 24 hours (scale bar=10 μm). (b) Codelivery of lysate and DNA by Lys-SNA (n=3, black bars) and Lys-Mix (n=3, green bars) to BMDCs in vitro after 1 and 24 hour incubations as assessed via flow cytometry. (c) Codelivery of lysate and DNA by Lys-SNA (n=3, black bars) and Lys-Mix (n=3, green bars) to lymphoid cells in vivo 2 and 24 hours following subcutaneous injection. Lymph nodes were isolated and CD11c+ lymphoid cells were analyzed by flow cytometry. Statistical analysis was performed using an ordinary one-way ANOVA, where “*” represents a p-value of <0.05, “**” represents a p-value of <0.01, and “***” represents a p-value of <0.001.

FIG. 6 shows the antitumor effects of Lys-SNA and Lys-Mix in vivo. (a) Antitumor efficacy of mice bearing orthotopic syngeneic (a) Py230 tumors, (b) Py8119 tumors, or (c) EMT6 tumors when administered Lys-SNA (black circles), Lys-Mix (green squares), or saline (white diamonds). Treatment initiation began at day 6 post-inoculation and was repeated on days 10 and 15. Statistical analysis was performed using an ordinary one-way ANOVA, where “*” represents a p-value of <0.05, “**” represents a p-value of <0.01, and “***” represents a p-value of <0.001.

FIG. 7 shows a Western blot of (a) lysates isolated from EMT6, Py230, and Py8119, and (b) lysates isolated from non-oxidized (left lane) and oxidized EMT6 cells (right lane).

FIG. 8 shows activation of BMDCs in vitro following incubation. Cells isolated from C57BL6 mice were purified and co-cultured with CpG-1826 and oxidized lysates (yellow bars), non-oxidized lysates (black bars), or no lysate (grey bars). After two days, DC activation was measured by flow cytometry for expression levels of (a) CD40, (b) CD80, (c) CD86, and (d) MHC-II. Statistical analysis was performed using an ordinary one-way ANOVA, where “*” represents a p-value of <0.05, “*” represents a p-value of <0.01, and “***” represents a p-value of <0.001.

FIG. 9 shows OxLys-SNA in vivo Analyses. (a) Antitumor efficacy and (b) corresponding survival curve of Balb/c mice bearing orthotopic syngeneic EMT6 tumors when administered OxLys-SNA (yellow circles), OxLys-Mix (grey squares), Lys-SNA (black circles), or saline (white diamonds). Treatment initiation began at day 6 post-inoculation and was repeated on days 10 and 15. Population of (d) cytotoxic CD8+ T cells and (e) MDSCs isolated from the tumor microenvironment of EMT6-bearing mice at day 11 post-inoculation, following treatment on days 6 and 15 with OxLys-SNA (yellow bars), Lys-SNA (black bars), OxLys-Mix (grey bars), or saline (white bars).

FIG. 10 shows (a) Individual tumor growth (spider) plots of animals administered OxLys-SNA from day 0 to day 25 post-inoculation. At day 20, all but 2 animals receiving OxLys-SNA treatment experienced complete tumor remission. (b) Spider plots of animals administered OxLys-SNA (yellow lines) or saline (black lines) out to day 50 post-inoculation. All saline-treated animals succumbed to tumor burden before the first OxLys-SNA treated animal. Blue box denotes the region in panel a.

FIG. 11 shows in vivo antitumor activity of L-SNAs in the Py8119 TNBC model. (a) Antitumor efficacy of “adjuvant only” L-SNAs as a function of liposome stability, following administration of saline (triangle), DOPC-SNAs (square), or DPPC-SNAs (circle). (b) Antitumor efficacy of L-SNAs encapsulating Py8119 lysates as a function of liposome stability. Animals were administered saline (triangle), DOPC-Lys-SNAs (square), or DPPC-Lys-SNAs (circle). (c) Antitumor efficacy of L-SNAs encapsulating oxidized Py8119 lysates as a function of liposome stability. Animals were administered saline (triangle), DOPC-OxLys-SNAs (square), or DPPC-OxLys-SNAs (circle). ((d) Comparison of tumor volume between DPPC-containing treatment groups at day 28 in the study. White dots represent individual animals in each group. Error bars represent standard error of the mean. Statistical analysis was performed using an unpaired t-test, where “*” represents a p-value of <0.05, “**” represents a p-value of <0.01, “***” represents a p-value of <0.001, and “ns” represents a p-value of >0.05.

### DETAILED DESCRIPTION

The present disclosure encompasses immunotherapeutic spherical nucleic acids (SNAs) (e.g., liposomal SNAs) that encapsulate lysates isolated from oxidized tumor cell and present immunostimulatory oligonucleotides (e.g., CpG-1826) as adjuvants on their surface. The resulting nanostructures (OxLys-SNAs) enhance the codelivery of adjuvant and antigen to immune cells as compared with simple mixtures of lysates with linear oligonucleotides, significantly increases the activation of dendritic cells relative to their non-oxidized counterparts.

An attractive alternative to single-antigen vaccines is to use lysates isolated from a patient's own tumor as the TAA source (12-18). Exploiting tumor cell lysates as antigens broadens the set of proteins that can be processed and targeted by immune cells—in principle, the entire tumor proteome can be accessed (18). Therefore, this also addresses several potential limitations of using a finite set of well-defined TAAs, including: 1) the challenge of identifying immunogenic epitopes from tumors, 2) epitope restriction by the major histocompatibility complex (MHC), and 3) loss of targeted antigens in tumors. However, direct vaccination using tumor lysates has been met with limited success due to low cellular uptake and bioavailability after injection, resulting in minimal immunogenicity (19). Oxidizing tumor cells prior to lysate isolation and preparation significantly increases immunogenicity when the lysates are utilized as the antigen source in DC vaccines (19-21). Importantly, protein chlorination by HOCl, an oxidant produced by neutrophils as part of the adaptive immune response, increases the immunogenicity of antigens by several-fold (22), potentially due to their increased proteolytic susceptibility (23). In addition, HOCl oxidation generates aldehyde-modified antigens that are more immunogenic than their unmodified counterparts (24). However, the underlying mechanism of how these changes facilitate antigen presentation and alter the tumor microenvironment (TME) remains unclear. Moreover, a major challenge in immunotherapeutic development is the selection of the appropriate vehicle for delivering both adjuvant and antigen (25), as the way components are formulated can significantly influence delivery to the immune system and thus, activation of immunostimulatory pathways (26, 27). Nanoscale therapeutics have shown promise in this regard, enhancing antigen-presenting cell (APC) activation over mixtures of adjuvant and antigen (28).

Spherical nucleic acids (SNAs) are a novel class of nucleic acids that exhibit completely different behavior from their linear analogs (29), including rapid cellular uptake without the use of ancillary transfection reagents (30). The SNA architecture is defined by a dense, highly oriented packing of nucleic acids into a spherical morphology, which imparts new chemical, biological, and physical properties to the nucleic acids from which SNAs are derived. To date, SNAs have been formed from a variety of nanoparticle cores, including gold and other inorganic nanoparticles (29-36), liposomes (37-41), polymers (42-44), and proteins (45). Liposomes are an especially attractive scaffold for SNA templating because the resulting systems are biodegradable and biocompatible, and liposomes are a validated, FDA-approved nanoscale formulation for drug delivery (46). In addition, the hollow core of liposomal SNAs can encapsulate TAAs and other cargo. Liposomal SNAs have been previously observed to initiate antigen presentation, activate immune cells, and induce production of proinflammatory cytokines for cancer treatment and other applications (39, 41, 47-49). In many of these examples, the sequence of the oligonucleotide shell is comprised of an unmethylated cytosine-guanosine sequence called CpG-1826. CpG-1826 mimics microbial genomes and acts as a pathogen-associated molecular pattern (PAMP) (50), which is recognized by toll-like receptor 9 (TLR9), a component of the innate immune system located in the endosome of antigen-presenting cells (APCs), including DCs (51).

The present disclosure provides SNAs containing tumor cell lysates that are used to develop potent nanoscale immunotherapeutics for the treatment of, in various aspects and embodiments, cancers without known TAAs, such as, without limitation, triple negative breast cancer (TNBC).

A “spherical nucleic acid (SNA)” as used herein comprises nucleic acids arranged around a nanoparticle core.

As used in this specification and the appended claims, the singular forms “a,” “an,” and “the” include plural reference unless the context clearly dictates otherwise.

The terms “polynucleotide” and “oligonucleotide” are interchangeable as used herein.

An “effective amount” or a “sufficient amount” of a substance is that amount necessary to effect beneficial or desired results, including clinical results, and, as such, an “effective amount” depends upon the context in which it is being applied. In the context of administering an antigenic composition, an effective amount contains sufficient antigen (e.g., a SNA comprising an oxidized tumor cell lysate of the disclosure) to elicit an immune response. An effective amount can be administered in one or more doses. Efficacy can be shown in an experimental or clinical trial, for example, by comparing results achieved with a substance of interest compared to an experimental control.

The term “dose” as used herein in reference to an antigenic composition refers to a measured portion of the antigenic composition taken by (administered to or received by) a subject at any one time.

The term “about” as used herein in reference to a value, encompasses from 90% to 110% of that value (e.g., a nanoparticle that is about 100 nanometers (nm) in diameter refers to a nanoparticle that is 90 nm to 110 nm in diameter).

The term “vaccination” as used herein refers to the introduction of vaccine into a body of an organism.

A “subject” is a living multi-cellular vertebrate organism. In the context of this disclosure, the subject can be an experimental subject, such as a non-human mammal (e.g., a mouse, a rat, or a non-human primate). Alternatively, the subject can be a human subject.

An “antigenic composition” is a composition of matter suitable for administration to a human or animal subject (e.g., in an experimental or clinical setting) that is capable of eliciting a specific immune response, e.g., against an antigen, such as a tumor associated antigen. As such, an antigenic composition includes one or more antigens (for example, tumor associated antigens) or antigenic epitopes. An antigenic composition can also include one or more additional components capable of eliciting or enhancing an immune response, such as an excipient, carrier, and/or adjuvant. In certain instances, antigenic compositions are administered to elicit an immune response that protects the subject against symptoms or conditions induced by an antigen. In some cases, symptoms or disease caused by an antigen is prevented (or reduced or ameliorated) by inhibiting expansion of cells associated with, e.g., a tumor. In the context of this disclosure, the term antigenic composition will be understood to encompass compositions that are intended for administration to a subject or population of subjects for the purpose of eliciting a protective or palliative immune response against a tumor associated antigen.

“Adjuvant” refers to a substance which, when added to a composition comprising an antigen, nonspecifically enhances or potentiates an immune response to the antigen in the recipient upon exposure. In any of the aspects or embodiments of the disclosure, the SNAs provided herein comprise immunostimulatory oligonucleotides (for example and without limitation, CpG-1826) as adjuvants and encapsulate lysates derived from tumor cells as antigens. Other common adjuvants that may be used in the compositions of the disclosure include suspensions of minerals (alum, aluminum hydroxide, aluminum phosphate) onto which an antigen is adsorbed; emulsions, including water-in-oil, and oil-in-water (and variants thereof, including double emulsions and reversible emulsions), liposaccharides, lipopolysaccharides, Pattern Recognition Receptor (PRR) agonists (e.g., NALP3. RIG-I-like receptors (RIG-I and MDA5), and various combinations of such components.

An “immune response” is a response of a cell of the immune system, such as a B cell, T cell, or monocyte, to a stimulus, such as an antigen (e.g., formulated as an antigenic composition or a vaccine). An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”). A “protective immune response” is an immune response that inhibits a detrimental function or activity of an antigen, or decreases symptoms (including death) that result from the antigen. A protective immune response can be measured, for example, by immune assays using a serum sample from an immunized subject for testing the ability of serum antibodies for inhibition of tumor cell expansion, such as: ELISA-neutralization assay, antibody dependent cell-mediated cytotoxicity assay (ADCC), complement-dependent cytotoxicity (CDC), antibody dependent cell-mediated phagocytosis (ADCP), enzyme-linked immunospot (ELISpot). In addition, vaccine efficacy can be tested by measuring the T cell response CD4+ and CD8+ after immunization, using flow cytometry (FACS) analysis or ELISpot assay. The protective immune response can be tested by measuring resistance to antigen challenge in vivo in an animal model. In humans, a protective immune response can be demonstrated in a population study, comparing measurements of symptoms, morbidity, mortality, etc. in treated subjects compared to untreated controls. Exposure of a subject to an immunogenic stimulus, such as an antigen (e.g., formulated as an antigenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an antigenic composition increases the magnitude of an antigen-specific response, (e.g., by increasing antibody titer and/or affinity, by increasing the frequency of antigen specific B or T cells, by inducing maturation effector function, or a combination thereof).

Spherical Nucleic Acids. Spherical nucleic acids (SNAs) comprise densely functionalized and highly oriented polynucleotides on the surface of a nanoparticle which can either be organic (e.g., a liposome), or polymeric (e.g., poly(lactic-co-glycolic acid) (PLGA), poly(acrylate), or poly(methacrylate). The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain (see, e.g., U.S. Patent Application Publication No. 2015/0031745, incorporated by reference herein in its entirety) and blood-tumor barriers as well as the epidermis(see, e.g., U.S. Patent Application Publication No. 2010/0233270, incorporated by reference herein in its entirety). As described herein, SNAs of the disclosure further comprise an oxidized tumor cell lysate encapsulated within the core of the SNA.

Nanoparticles are therefore provided which are functionalized to have a polynucleotide attached thereto. In general, nanoparticles contemplated include any compound or substance with a high loading capacity for a polynucleotide as described herein, including for example and without limitation, a liposomal particle, a polymer-based particle (e.g., a poly (lactic-co-glycolic acid) (PLGA) particle), or a dendrimer (organic versus inorganic).

Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles.

Liposomal particles, for example as disclosed in International Patent Application No. PCT/US2014/068429 (incorporated by reference herein in its entirety, particularly with respect to the discussion of liposomal particles) are also contemplated by the disclosure. Hollow particles, for example as described in U.S. Patent Publication Number 2012/0282186 (incorporated by reference herein in its entirety) are also contemplated herein. Liposomal particles of the disclosure have at least a substantially spherical geometry, an internal side and an external side, and comprise a lipid bilayer. The lipid bilayer comprises, in various embodiments, a plurality of lipid groups wherein at least one lipid group is of the phosphocholine family of lipids or the phosphoethanolamine family of lipids. While not meant to be limiting, the at least one lipid group is 1,2-dioleoyl-sn-glycero-3-phosphocholine (DOPC), 1,2-dimyristoyl-sn-phosphatidylcholine (DMPC), 1,2-dihexadecanoyl-sn-glycero-3-phosphatidylcholine (DPPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1-oleoyl-2-hydroxy-sn-glycero-3-phosphoethanolamine, 1-stearoyl-2-hydroxy-sn-glycero-3-phosphoethanolamine, 1,2-distearoyl-sn-glycero-3-phosphoethanolamine, and 1,2-dielaidoyl-sn-glycero-3-phosphoethanolamine, 1,2-dioleoyl-sn-glycero-3-phosphoethanolamine-N-[azido(polyethylene glycol)] (DOPE-PEG-azide), 1,2-dioleoyl-sn-glycero-3-phosphoethanolamine-N-[maleimide(polyethylene glycol)] (DOPE-PEG-maleimide), 1,2-dipalmitoyl-sn-glycero-3-phosphoethanolamine-N-[azido(polyethylene glycol)] (DPPE-PEG-azide), 1,2-dipalmitoyl-sn-glycero-3-phosphoethanolamine-N-[maleimide(polyethylene glycol)] (DPPE-PEG-maleimide), 1,2-distearoyl-sn-glycero-3-phosphoethanolamine-N-[azido(polyethylene glycol)] (DSPE-PEG-azide), and 1,2-distearoyl-sn-glycero-3-phosphoethanolamine-N-[maleimide(polyethylene glycol)] (DSPE-PEG-maleimide), 1,2-dihexadecanoyl-sn-glycero-3-phosphoethanolamine (DPPE), or a combination thereof. In some embodiments, the plurality of lipid groups comprises or consists of 1,2-dipalmitoyl-sn-glycero-3-phosphocholine (DPPC). In some embodiments, the plurality of lipid groups comprises or consists of 1,2-dioleoyl-sn-glycero-3-phosphocholine (DOPC).

Nanoparticles can range in size from about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In other aspects, the disclosure provides a plurality of nanoparticles, each nanoparticle having a substantially spherical geometry comprising an oligonucleotide conjugated thereto, wherein the oligonucleotide is a Toll-Like Receptor (TLR) agonist, and wherein an oxidized tumor cell lysate is encapsulated within the nanoparticle. In these aspects, the size of the plurality of nanoparticles is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of nanoparticles) of the nanoparticles is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the nanoparticles, for example, optical properties or the amount of surface area that can be functionalized as described herein. In further embodiments, a plurality of SNAs (e.g., liposomal particles) is produced and the SNAs in the plurality have a mean diameter of less than or equal to about 150 nanometers (e.g., about 10 nanometers to about 150 nanometers), or less than or equal to about 100 nanometers (e.g., about 10 nanometers to about 100 nanometers, or less than or equal to about 80 nanometers (e.g., about 10 nanometers to about 80 nanometers). In further embodiments, the nanoparticles in the plurality created by a method of the disclosure have a diameter or mean diameter of less than or equal to about 20 nanometers, or less than or equal to about 25 nanometers, or less than or equal to about 30 nanometers, or less than or equal to about 35 nanometers, or less than or equal to about 40 nanometers, or less than or equal to about 45 nanometers, or less than or equal to about 50 nanometers, or less than or equal to about 55 nanometers, or less than or equal to about 60 nanometers, or less than or equal to about 65 nanometers, or less than or equal to about 70 nanometers, or less than or equal to about 75 nanometers, or less than or equal to about 80 nanometers, or less than or equal to about 85 nanometers, or less than or equal to about 90 nanometers, or less than or equal to about 95 nanometers, or less than or equal to about 100 nanometers, or less than or equal to about 100 nanometers, or less than or equal to about 120 nanometers, or less than or equal to about 130 nanometers, or less than or equal to about 140 nanometers, or less than or equal to about 150 nanometers. It will be understood that the foregoing diameters of nanoparticles can apply to the diameter of the nanoparticle itself or to the diameter of the nanoparticle and oligonucleotides associated therewith.

Oligonucleotides. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotides or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deazaadenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof, generally comprise a polynucleotide from about 5 nucleotides to about 100 nucleotides in length. More specifically, nanoparticles are functionalized with a polynucleotide that is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated.

In some embodiments, the polynucleotide attached to a nanoparticle is DNA. When DNA is attached to the nanoparticle, the DNA is in some embodiments comprised of a sequence that is sufficiently complementary to a target region of a polynucleotide such that hybridization of the DNA polynucleotide attached to a nanoparticle and the target polynucleotide takes place, thereby associating the target polynucleotide to the nanoparticle. The DNA in various aspects is single stranded or double-stranded, as long as the double-stranded molecule also includes a single strand region that hybridizes to a single strand region of the target polynucleotide. In some aspects, hybridization of the polynucleotide functionalized on the nanoparticle can form a triplex structure with a double-stranded target polynucleotide. In another aspect, a triplex structure can be formed by hybridization of a double-stranded oligonucleotide functionalized on a nanoparticle to a single-stranded target polynucleotide. In some embodiments, the disclosure contemplates that a polynucleotide attached to a nanoparticle is RNA. The RNA can be either single-stranded or double-stranded (e.g., siRNA), so long as it is able to hybridize to a target polynucleotide. In various embodiments, the polynucleotide attached to a nanoparticle is 100% complementary to a target polynucleotide, i.e., a perfect match, while in other aspects, the polynucleotide attached to a nanoparticle is at least about 95% complementary to the target polynucleotide over the length of the polynucleotide attached to a nanoparticle, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, or at least about 20% complementary to the target polynucleotide over the length of the polynucleotide attached to a nanoparticle.

In some aspects, multiple polynucleotides are functionalized to a nanoparticle. In various aspects, the multiple polynucleotides each have the same sequence, while in other aspects one or more polynucleotides have a different sequence. In further aspects, multiple polynucleotides are arranged in tandem and are separated by a spacer. Spacers are described in more detail herein below.

Polynucleotide attachment to a nanoparticle. Polynucleotides contemplated for use in the methods include those bound to the nanoparticle through any means (e.g., covalent or non-covalent attachment). Regardless of the means by which the polynucleotide is attached to the nanoparticle, attachment in various aspects is effected through a 5′ linkage, a 3′ linkage, some type of internal linkage, or any combination of these attachments. In some embodiments, the polynucleotide is covalently attached to a nanoparticle. In further embodiments, the polynucleotide is non-covalently attached to a nanoparticle. An oligonucleotide of the disclosure comprises, in various embodiments, a tocopherol, a cholesterol moiety, DOPE-butamide-phenylmaleimido, or lyso-phosphoethanolamine-butamide-phenylmaleimido. In some embodiments, the cholesterol is cholesteryl-triethyleneglycol (cholesteryl-TEG). In some embodiments, tocopherol is chosen from the group consisting of a tocopherol derivative, alpha-tocopherol, beta-tocopherol, gamma-tocopherol and delta-tocopherol. See also U.S. Patent Application Publication No. 2016/0310425, incorporated by reference herein in its entirety.

Methods of attachment are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety. Methods of associating polynucleotides with a liposomal particle are described in PCT/US2014/068429, which is incorporated by reference herein in its entirety.

Spacers. In certain aspects, functionalized nanoparticles are contemplated which include those wherein an oligonucleotide is attached to the nanoparticle through a spacer. “Spacer” as used herein means a moiety that does not participate in modulating gene expression per se but which serves to increase distance between the nanoparticle and the functional oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof. In various embodiments, an oligonucleotide comprises 1, 2, 3, 4, 5, or more spacer (e.g., Spacer-18 (hexaethyleneglycol)) moieties.

As a result of the binding of the spacer to the nanoparticles, the polynucleotide is spaced away from the surface of the nanoparticles and is more accessible for association with its target. In various embodiments, the length of the spacer is or is equivalent to at least about 5 nucleotides, 5-10 nucleotides, 10 nucleotides, 10-30 nucleotides, or even greater than 30 nucleotides. The spacer may have any sequence which does not interfere with the ability of the polynucleotides to become bound to the nanoparticles or to the target. In certain aspects, the bases of the polynucleotide spacer are all adenylic acids, all thymidylic acids, all cytidylic acids, all guanylic acids, all uridylic acids, or all some other modified base.

Nanoparticle surface density. A surface density adequate to make the nanoparticles stable and the conditions necessary to obtain it for a desired combination of nanoparticles and polynucleotides can be determined empirically. Generally, a surface density of at least about 2 pmoles/cm² will be adequate to provide stable nanoparticle-oligonucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Alternatively, the density of polynucleotide on the surface of the SNA is measured by the number of polynucleotides on the surface of a SNA. With respect to the surface density of polynucleotides on the surface of a SNA of the disclosure, it is contemplated that a SNA as described herein comprises about 1 to about 25,000 oligonucleotides on its surface. In various embodiments, a SNA comprises about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20 oligonucleotides on its surface. In some embodiments, a SNA comprises about 80 to about 140 oligonucleotides on its surface. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 polynucleotides on its surface. In further embodiments, a SNA consists of 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 polynucleotides on its surface. In some embodiments, a liposomal SNA (which may, in various embodiments, be about or less than about 150 nanometers in diameter or about or less than about 100 nanometers in diameter or about or less than about 80 nanometers in diameter or about or less than about 70 nanometers in diameter) comprises about 10 to about 1,000 oligonucleotides or about 10 to about 40 oligonucleotides on its surface. In further embodiments, PLGA SNA (which may, in various embodiments, be less than about 100 nanometers in diameter or less than about 80 nanometers in diameter) comprises about 10 to about 800 oligonucleotides on its surface.

### Methods of Making Spherical Nucleic Acids (SNAs)

According to the present disclosure, the way in which lysates are processed, packaged, and presented to the immune cell is a critical determinant of the therapeutic potential of lysate-based immunotherapeutics. Thus, the disclosure provides methods of making a nanoparticle having a substantially spherical geometry comprising an oligonucleotide conjugated thereto, wherein the oligonucleotide is a Toll-Like Receptor (TLR) agonist, and wherein an oxidized tumor cell lysate is encapsulated within the nanoparticle. The method comprises exposing a tumor cell to an oxidizing agent to produce an oxidized tumor cell; then isolating lysate from the oxidized tumor cell; then contacting a lipid film with the lysate to produce a small unilamellar vesicle (SUV) comprising the lysate encapsulated therein; and then adding an oligonucleotide to the SUV to make the liposomal nanoparticle. The disclosure also specifically contemplates nanoparticles produced by the foregoing method.

The tumor cells are oxidized prior to lysate isolation, preparation, and encapsulation within a nanoparticle core. The tumor cell is oxidized via exposure to an oxidizing agent. The tumor cell is exposed to the oxidizing agent for about 30 minutes to about 2 hours, or about 30 minutes to about 1 hour at 25-37° C. In some embodiments, the tumor cell is exposed to the oxidizing agent for about 1 hour at 37° C. The oxidizing agent is in various embodiments, hypochlorous acid (HOCl), hydrogen peroxide, sodium hypochlorite, sodium chlorite, nitric acid, sulfur, or a combination thereof. In various embodiments, the tumor cell is exposed to about 10 μM to about 100 μM, or about 10 μM to about 90 μM, or about 10 μM to about 80 μM, or about 10 μM to about 70 μM, or about 10 μM to about 60 μM, or about 10 μM to about 50 μM, or about 10 μM to about 40 μM, or about 10 μM to about 30 μM, or about 10 μM to about 20 μM of the oxidizing agent. In further embodiments, the tumor cell is exposed to about 10 μM, 20 μM, 30 μM, 40 μM, 50 μM, 60 μM, 70 μM, 80 μM, 90 μM, or 100 μM of the oxidizing agent. In further embodiments, the tumor cell is exposed to at least about 10 μM, 20 μM, 30 μM, 40 μM, 50 μM, 60 μM, 70 μM, 80 μM, 90 μM, or 100 μM of the oxidizing agent. In further embodiments, the tumor cell is exposed to less than about 10 μM, 20 μM, 30 μM, 40 μM, 50 μM, 60 μM, 70 μM, 80 μM, 90 μM, or 100 μM of the oxidizing agent. In some embodiments, the tumor cell is exposed to about 60 μM of HOCl for about 1 hour at 37° C. In various embodiments, the tumor cell is a breast cancer cell, peritoneum cancer cell, cervical cancer cell, colon cancer cell, rectal cancer cell, esophageal cancer cell, eye cancer cell, liver cancer cell, pancreatic cancer cell, larynx cancer cell, lung cancer cell, skin cancer cell, ovarian cancer cell, prostate cancer cell, stomach cancer cell, testicular cancer cell, thyroid cancer cell, brain cancer cell, or a combination thereof. In some embodiments, the tumor cell is a triple negative breast cancer (TNBC) cell.

The tumor cell lysate is then isolated from the oxidized tumor cell and is contacted with a lipid film to produce a small unilamellar vesicle (SUV) comprising the lysate encapsulated therein. As described herein, the oxidized tumor cell lysate may also be encapsulated within a poly(lactic-co-glycolic acid) (PLGA) nanoparticle, poly(acrylate) nanoparticle, or a poly(methacrylate) nanoparticle. In various embodiments, the amount of tumor cell lysate that is encapsulated within a nanoparticle is about 5 μg to about 150 μg tumor cell lysate. In further embodiments, the amount of tumor cell lysate that is encapsulated within a nanoparticle is about 5 μg to about 140 μg tumor cell lysate, or about 5 μg to about 130 μg tumor cell lysate, or about 5 μg to about 120 μg tumor cell lysate, or about 5 μg to about 110 μg tumor cell lysate, or about 5 μg to about 100 μg tumor cell lysate, or about 5 μg to about 90 μg tumor cell lysate, or about 5 μg to about 80 μg tumor cell lysate, or about 5 μg to about 70 μg tumor cell lysate, or about 5 μg to about 60 μg tumor cell lysate, or about 5 μg to about 50 μg tumor cell lysate, or about 5 μg to about 40 μg tumor cell lysate, or about 5 μg to about 30 μg tumor cell lysate or about 5 μg to about 20 μg tumor cell lysate, or about 5 μg to about 10 μg tumor cell lysate. In still further embodiments, the amount of tumor cell lysate that is encapsulated within a nanoparticle is or is about 5 μg, 10 μg, 20 μg, 30 μg, 40 μg, 50 μg, 60 μg, 70 μg, 80 μg, 90 μg, 100 μg, 110 μg, 120 μg, 130 μg, 140 μg, or 150 μg tumor cell lysate. In further embodiments, the amount of tumor cell lysate that is encapsulated within a nanoparticle is at least about 5 μg, 10 μg, 20 μg, 30 μg, 40 μg, 50 μg, 60 μg, 70 μg, 80 μg, 90 μg, 100 μg, 110 μg, 120 μg, 130 μg, 140 μg, or 150 μg tumor cell lysate. In some embodiments, the amount of tumor cell lysate that is encapsulated within a nanoparticle is less than about 5 μg, 10 μg, 20 μg, 30 μg, 40 μg, 50 μg, 60 μg, 70 μg, 80 μg, 90 μg, 100 μg, 110 μg, 120 μg, 130 μg, 140 μg, or 150 μg tumor cell lysate.

Next, an oligonucleotide is added to the nanoparticle (e.g., SUV) that has tumor cell lysate encapsulated therein. In various embodiments, the amount of oligonucleotide that is added to the nanoparticle that has tumor cell lysate encapsulated therein is about 0.5 nmol to about 25 nmol. In further embodiments, the amount of oligonucleotide that is added to the nanoparticle that has tumor cell lysate encapsulated therein is about 0.5 nmol to about 20 nmol, or about 0.5 nmol to about 15 nmol, or about 0.5 nmol to about 10 nmol, or about 1 nmol to about 10 nmol, or about 1 nmol to about 8 nmol, or about 1 nmol to about 6 nmol. In still further embodiments, the amount of oligonucleotide that is added to the nanoparticle that has tumor cell lysate encapsulated therein is or is about 0.5 nmol, 1 nmol, 1.5 nmol, 2 nmol, 2.5 nmol, 3 nmol, 3.5 nmol, 4 nmol, 4.5 nmol, 5 nmol, 6.5 nmol, 7 nmol, 7.5 nmol, 8 nmol, 8.5 nmol, 9 nmol, 9.5 nmol, 10 nmol, 11 nmol, 12 nmol, 13 nmol, 14 nmol, 15 nmol, 16 nmol, 17 nmol, 18 nmol, 19 nmol, 20 nmol, 21 nmol, 22 nmol, 23 nmol, 24 nmol, or 25 nmol. In further embodiments, the amount of oligonucleotide that is added to the nanoparticle that has tumor cell lysate encapsulated therein is at least about 0.5 nmol, 1 nmol, 1.5 nmol, 2 nmol, 2.5 nmol, 3 nmol, 3.5 nmol, 4 nmol, 4.5 nmol, 5 nmol, 6.5 nmol, 7 nmol, 7.5 nmol, 8 nmol, 8.5 nmol, 9 nmol, 9.5 nmol, 10 nmol, 11 nmol, 12 nmol, 13 nmol, 14 nmol, 15 nmol, 16 nmol, 17 nmol, 18 nmol, 19 nmol, 20 nmol, 21 nmol, 22 nmol, 23 nmol, 24 nmol, or 25 nmol. In further embodiments, the amount of oligonucleotide that is added to the nanoparticle that has tumor cell lysate encapsulated therein is less than about 0.5 nmol, 1 nmol, 1.5 nmol, 2 nmol, 2.5 nmol, 3 nmol, 3.5 nmol, 4 nmol, 4.5 nmol, 5 nmol, 6.5 nmol, 7 nmol, 7.5 nmol, 8 nmol, 8.5 nmol, 9 nmol, 9.5 nmol, 10 nmol, 11 nmol, 12 nmol, 13 nmol, 14 nmol, 15 nmol, 16 nmol, 17 nmol, 18 nmol, 19 nmol, 20 nmol, 21 nmol, 22 nmol, 23 nmol, 24 nmol, or 25 nmol. In some embodiments, the amount of oligonucleotide that is added to the nanoparticle that has tumor cell lysate encapsulated therein is 5 nmol. In some embodiments, the oligonucleotide is a Toll-Like Receptor (TLR) agonist. In further embodiments, the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLRS) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof. In some embodiments, the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof.

In various embodiments, the foregoing methods produce a nanoparticle that comprises a ratio of oligonucleotide to tumor cell lysate that is about 0.5 nmol to about 25 nmol: about 5 μg to about 150 μg. In further embodiments, the nanoparticle comprises a ratio of oligonucleotide to tumor cell lysate that is about 5 nmol oligonucleotide:20 μg tumor cell lysate.

The disclosure also specifically contemplates nanoparticles having the foregoing features.

### Compositions

The disclosure includes compositions that comprise a nanoparticle having a substantially spherical geometry comprising an oligonucleotide conjugated thereto (i.e., a spherical nucleic acid (SNA)), wherein the oligonucleotide is a Toll-Like Receptor (TLR) agonist, and wherein an oxidized tumor cell lysate is encapsulated within the nanoparticle. In some embodiments, the composition is an antigenic composition. In some embodiments, the composition further comprises a pharmaceutically acceptable carrier. The term “carrier” refers to a vehicle within which the nanoparticle as described herein is administered to a mammalian subject. The term carrier encompasses diluents, excipients, adjuvants and combinations thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances that may enhance vaccine stability and include but are not limited to polymers (e.g., polyethylene glycol), carbohydrates (e.g., starch, glucose, lactose, sucrose, or cellulose), and alcohols (e.g., glycerol, sorbitol, or xylitol).

Adjuvants include vaccine delivery systems (e.g., emulsions, microparticles, immune stimulating complexes (ISCOMS), or liposomes) that target associated antigens to antigen presenting cells (APC); and immunostimulatory adjuvants.

### Methods of Inducing an Immune Response

The disclosure includes methods for eliciting an immune response in a subject in need thereof, comprising administering to the subject an effective amount of an antigenic composition comprising one or more of the SNA comprising an oxidized tumor cell lysate as described herein. Unless otherwise indicated, the antigenic composition is an immunogenic composition.

The immune response raised by the methods of the present disclosure generally includes an antibody response, preferably a neutralizing antibody response, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the SNA comprising an oxidized tumor cell lysate as disclosed herein generates an immune response that recognizes, and preferably ameliorates and/or neutralizes, cancer as described herein. Methods for assessing antibody responses after administration of an antigenic composition (immunization or vaccination) are known in the art and/or described herein. In some embodiments, the immune response comprises a T cell-mediated response (e.g., peptide-specific response such as a proliferative response or a cytokine response). In preferred embodiments, the immune response comprises both a B cell and a T cell response. Antigenic compositions can be administered in a number of suitable ways, such as intramuscular injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Antigenic compositions may be used to treat both children and adults, including pregnant women. Thus a subject may be less than 1 year old, 1-5 years old, 5-15 years old, 15-55 years old, or at least 55 years old. Preferred subjects for receiving the vaccines are the elderly (e.g., >55 years old, >60 years old, preferably >65 years old), and the young (e.g., <6 years old, 1-5 years old, preferably less than 1 year old). Additional subjects for receiving the vaccines or compositions of the disclosure include naïve (versus previously infected) subjects, currently infected subjects, or immunocompromised subjects.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, or a mucosal prime and parenteral boost. Administration of more than one dose (typically two doses) is particularly useful in immunologically naive subjects or subjects of a hyporesponsive population (e.g., diabetics, or subjects with chronic kidney disease (e.g., dialysis patients)). Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). Preferably multiple doses are administered from one, two, three, four or five months apart. Antigenic compositions of the present disclosure may be administered to patients at substantially the same time as (e.g., during the same medical consultation or visit to a healthcare professional) other vaccines.

In general, the amount of SNA comprising an oxidized tumor cell lysate in each dose of the antigenic composition is selected as an amount effective to induce an immune response in the subject, without causing significant, adverse side effects in the subject. Preferably the immune response elicited includes: neutralizing antibody response; antibody dependent cell-mediated cytotoxicity (ADCC); antibody cell-mediated phagocytosis (ADCP); complement dependent cytotoxicity (CDC); T cell-mediated response such as CD4+, CD8+, or a protective antibody response. Protective in this context does not necessarily require that the subject is completely protected against infection. A protective response is achieved when the subject is protected from developing symptoms of disease. As described above, the immune response generated by the SNA comprising an oxidized tumor cell lysate as disclosed herein generates an immune response that recognizes, and preferably ameliorates and/or neutralizes, cancer as described herein.

### EXAMPLES

The following examples demonstrate that SNAs containing tumor cell lysates can be used to develop potent nanoscale immunotherapeutics for the treatment of cancers without known TAAs, such as triple negative breast cancer (TNBC).

Cancers without identified tumor-associated antigens (TAAs), such as triple negative breast cancer (TNBC), remain challenging immunotherapeutic targets. Herein, the synthesis and evaluation of immunotherapeutic liposomal spherical nucleic acids (SNAs) for the treatment of TNBC is described. The SNAs comprise immunostimulatory oligonucleotides (CpG-1826) as adjuvants and encapsulate lysates derived from TNBC cell lines as antigens. The resulting nanostructures (Lys-SNAs) enhance the codelivery of adjuvant and antigen to immune cells when compared to simple mixtures of lysates with linear oligonucleotides both in vitro and in vivo, and reduce tumor growth relative to simple mixtures of lysate and CpG-1826 (Lys-Mix) in both Py230 and Py8119 orthotopic syngeneic mouse models of TNBC. Furthermore, oxidizing TNBC cells, prior to lysis and incorporation into SNAs (OxLys-SNAs), significantly increases the activation of dendritic cells relative to their non-oxidized counterparts. When administered peritumorally in vivo in the EMT6 mouse mammary carcinoma model, OxLys-SNAs significantly increase the population of cytotoxic CD8+ T cells and simultaneously decrease the population of myeloid derived suppressor cells (MDSCs), when compared to Lys-SNAs and simple mixtures of oxidized lysates with immunostimulatory oligonucleotides. Importantly, animals treated with OxLys-SNAs exhibit significant antitumor activity and prolonged survival relative to all other treatment groups, and resist tumor re-challenge. Together, these results showed that the way lysates are processed and packaged has a profound impact on their immunogenicity and therapeutic efficacy. Moreover, this work points toward the use of oxidized tumor cell lysate-loaded SNAs as a potent new class of immunotherapeutics for cancers without identified tumor-associated antigens.

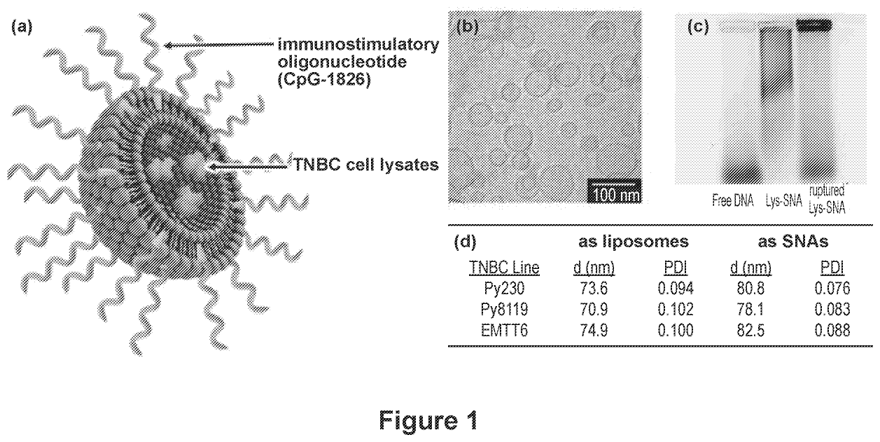

Accounting for the majority of all breast cancer-related mortality (52, 53), TNBC is a highly heterogeneous and aggressive disease that lacks functional expression of both estrogen and progesterone receptors and no overexpression of human epidermal growth factor receptor 2 (HER2) protein (52-55). Paradoxically, TNBC primary tumors often initially respond well to chemotherapy; yet, there is a high incidence of relapse and metastasis. The early and aggressive nature of TNBC recurrence is exemplified by significantly decreased rates of progression-free and three-year overall survival vs. other breast cancer subtypes (53, 56, 57), necessitating the development of new and effective treatment options. In an effort to explore the potential of SNAs as a therapeutic for treating TNBC, liposomal SNAs were synthesized that encapsulate lysates derived from TNBC cell lines in their core and present CpG-1826 on their surfaces (Lys-SNAs, FIG. 1), as well as analogs that contain lysates from TNBC cells that were oxidized with hypochlorous acid (HOCl) prior to lysis (OxLys-SNAs), and evaluated their immunomodulatory activity and antitumor properties in syngeneic, orthotopic mouse models of TNBC.

### Example 1

### Materials and Methods

Generation and Characterization of Cell Lysates. EMT6 cell lines were obtained from ATCC and grown in Minimum Essential Media (MEM) supplemented with 10% heat-inactivated fetal bovine serum and 1% penicillin-streptomycin. Py230 and Py8119 were grown in F-12K Medium (ATCC), 5% fetal bovine serum, 0.1% MITO+ serum extender (Corning), 2.5 μg/mL amphotericin B (Gibco), and 50 μg/mL gentamycin (Gibco). All lysates were prepared from cells under passage number six. For lysate preparation, cells were trypsinized, washed, collected and resuspended at 10⁶ cells/mL in Dulbecco's phosphate buffered saline (DPBS) then subjected to five freeze-thaw cycles in liquid nitrogen and a 37° C. water bath. Cellular debris was removed by centrifugation at 10,000 RCF for 10 minutes and the supernatant was then collected as the protein lysate. Total protein concentration was measured using the bicinchoninic acid (BCA) assay with albumin as the protein standard (Pierce, ThermoFisher Scientific). Protein content was characterized using 4-12% SDS-PAGE electrophoresis at 100 V for 1 hour and loading 20 μg total protein.

To prepare oxidized lysates, EMT6 cells were grown to confluence in a petri dish. Cells were washed with DPBS (3×) and then incubated with 60 μM hypochlorous acid (HOCl) in DPBS for 1 hour at 37° C. Following the incubation, cells were collected and washed with DPBS to remove any unreacted HOCl. Following centrifugation at 500 RCF for 5 minutes, cells were resuspended in DPBS at a density of 1×10⁷ cells/mL. Cells were subjected to 5 freeze-thaw cycles using liquid nitrogen and a 37° C. water bath, followed by centrifugation for 10 minutes at 10,000 RCF. The soluble fraction was collected as the oxidized lysate.

Purified cell lysates were fluorophore-labeled for in vitro and in vivo uptake experiments. One milligram each of Oregon Green 488-NHS (Thermo Fisher) and fluorescein-5-maleimide (Thermo Fisher) dye were incubated with 1 mg/mL lysate in phosphate buffered saline (PBS, pH 7.5) for 16 hours at 4° C. The unreacted dye was removed by washing the lysate with 10 mL of PBS 10 times using 50 kDa cutoff centrifugation filters (4000 g, 10 minutes). Fluorophore-labeled lysate was subsequently used to make lysate-SNAs.

DNA Synthesis. Cholesteryl-modified CpG-1826 (5′-TCC ATG ACG TTC CTG ACG TT (Spacer-18 (hexaethyleneglycol))₂ Chol-3′) (SEQ ID NO: 2), as well as Cholesteryl-Cy5-modified CpG-1826 (5′-TCC ATG ACG TTC CTG ACG TT-Cy5-(Spacer-18 (hexaethyleneglycol))₂ Chol-3′) (SEQ ID NO: 3) were synthesized with phosphorothioate (PS) backbones via automated solid-phase DNA synthesis using an MerMade 12 Synthesizer (Bioautomation), using DCI as an activator and 3-((Dimethylamino-methylidene)amino)-3H-1,2,4-dithiazole-3-thione as the sulfurizing agent. Following synthesis, DNA strands were cleaved from solid support via overnight incubation with 30% ammonium hydroxide at RT. Excess ammonia was removed through evaporation under nitrogen, and oligonucleotides purified using HPLC (Agilent) on a C4 or C18 column, using a gradient of triethylammonium acetate (TEAA) and acetonitrile (10% to 100% acetonitrile) over 30 minutes. Purified oligonucleotides were collected and lyophilized. Powdered oligonucleotides were reconstituted in 5 mL acetic acid and incubated at RT for 1 hour, then extracted with ethyl acetate (7 mL, 3×). The purified, deprotected DNA was then lyophilized, resuspended in 1 mL deionized water, and analyzed by MALDI-TOF and native gel electrophoresis.

Synthesis of Lysate-Loaded SNAs. Tumor cell lysate (either oxidized or not) was encapsulated within DOPC liposomes using the thin-film rehydration method (56). Solutions were adjusted to 1 mg/mL (with respect to protein concentration) in phosphate buffered saline (PBS), which was used to rehydrate 5 mg DOPC for 1 hour at room temperature. After the rehydration period, liposomes were formed through five freeze-thaw cycles, using liquid nitrogen and sonication in a 37° C. water bath. Liposomes were then diluted with PBS such that the highest concentration of lipid was no greater than 2 mg/mL lipid for extrusion, as measured by the commercially available phosphatidylcholine (PC) assay (Sigma). Liposome size was controlled through sequential high-pressure extrusion using polycarbonate filters (T&T Scientific) with pore sizes of 200, 100, 80, and 50 nm. Liposomes were passed through each filter size ten times. Following the final extrusion, tangential flow filtration (TFF) with a pore size of 500 kDa (Spectrum) was used to remove any non-encapsulated proteins and the sample was repeatedly washed with PBS until no protein was detected in the flow-through, as monitored by measuring the absorption of the flow-through at 280 nm with UV-vis spectroscopy (Cary) and BCA assay. The amount of protein encapsulated within the liposomes was measured using the BCA assay after disruption of the liposome with 1% SDS to release encapsulated protein. The phospholipid concentration was measured using commercially available PC assay kit.

To form SNAs, cholesterol-terminated oligonucleotides (3′) were embedded into the outer membrane of the liposomes by mixing 20 μM oligonucleotides with a solution of liposomes at 1.63 mM lipid at 25° C. overnight. The oligonucleotide concentration was determined by measuring the absorption at 260 nm with UV-vis. The resulting SNAs (both OxLys-SNAs and Lys-SNAs) were then concentrated to 20 μM by DNA using centrifugation filter units (Milipore), which also removed any unbound DNA. The resulting structures were analyzed by zeta potential (Malvern Zetasizer), gel electrophoresis, and DLS (FIG. 1).

Characterization of Lysate-Loaded SNAs. Lysate-loaded SNAs were characterized using cryo-TEM, gel electrophoresis, DLS (FIG. 1d), and zeta potential (Table 1). CryoEM samples were prepared by FEI Vitrobot Mark III by dropping 4 μL on a 200-mesh copper TEM grid with lacey carbon film, blotted for 5 seconds, and then plunged into liquid ethane before transfer to a cryo holder and storage in liquid nitrogen prior to imaging. cryoEM imaging was performed using a Hitachi HT7700 transmission electron microscope with a Gatan cryo-transfer holder under 120 kV accelerating voltage, and images were taken with a Gatan imaging camera at 30,000× magnification. Confirmation of DNA loading was done using gel electrophoresis, DLS, and zeta potential measurements. Cy5-labeled, cholesteryl-modified oligonucleotides, Cy5-labeled Lys-SNAs, and Cy5-labeled Lys-SNAs that had been incubated with Triton-X to dissociate liposomes (50 pmol each) were loaded into a 1% agarose gel on ice and ran at 100 V for 45 minutes (FIG. 1c).

TABLE 1
Zeta potential analyses of Lys-SNAs and OxLys-SNAs.

| | as liposomes | | as SNAs | |
|---|---|---|---|---|
| | zeta potential | | zeta potential | |
| TNBC Line | (mV) | St Dev. | (mV) | St Dev. |
| Py230 | −7.05 | 1.42 | −23.45 | 2.75 |
| Py8119 | −4.28 | 3.80 | −27.83 | 1.68 |
| EMT6 | −5.37 | 1.14 | −28.01 | 1.52 |
| Ox-EMT6 | −5.66 | 1.84 | −35.25 | 0.08 |

Uptake of Lys-SNAs and Lys-Mix by BMDCs in vitro. Bone marrow was isolated from femurs of Balb/C or C57BL/6 mice and cultured in RPMI-1640 supplemented with 10% heat-inactivated fetal bovine serum, 1% penicillin-streptomycin, and 20 ng/mL GM-CSF. Media was replenished on day three and cells were harvested on day six. SNAs were prepared by encapsulating AlexaFluor488-labeled lysate within the liposomal core and functionalizing Cy5-labeled DNA using the same methods as described above. Fluorophore-labeled SNAs were used to measure the uptake of particles in BMDCs. BMDCs were added to 24-well plates at 500,000 cells per well, and immediately treated with 1 μM Cy5-labeled DNA and AlexaFluor488-labeled lysate, or doubly-labeled SNAs for 1 or 24 hours. Cells were washed, fixed with 4% paraformaldehyde for 10 minutes at room temperature and either resuspended in DPBS for flow cytometry (BD LSRFortessa), or stained with DAPI and imaged with confocal microscopy (Zeiss LSM 800) using a 63× objective.

Uptake of Lys-SNAs and Lys-Mix by Lymphoid Cells in vivo. Female C57BL/6 mice (n=3, 8-10 weeks old) were injected subcutaneously (flank) with 2004 of 50 uM fluorophore-labeled Lys-SNAs or a mixture of CpG-1826 and lysate (Lys-Mix). Mice were euthanized 2 or 24 hours post-injection and draining lymph nodes were excised. Lymph nodes were dissociated into single cells using a cell strainer. Single cell suspensions were stained with antibodies for CD11c (PE-Cy7) as well as a live/dead stain and analyzed using flow cytometry.

Lys-SNA Antitumor Efficacy. Female mice (age 8-10 weeks) were inoculated with 1×10⁶ TNBC cells (C57Bl/6 mice for Py230 and Py8119; Balb/C mice for EMT6) via subcutaneous injection into the right inguinal mammary fat pad. On days 6, 10, and 15, animals were administered Lys-SNA, Lys-Mix, or saline (n=5 per group) via peritumoral injection (50 μM, 200 μL). Tumor volumes were calculated by measuring the length and width with calipers and applying the formula V=L×W×W/2. Studies were stopped and animals sacrificed when tumor burden of saline-treated animals exceeded 1200 mm³.

OxLys-SNA Antitumor Efficacy. Female Balb/C mice (age 8-10 weeks) were inoculated with 1×10⁶ EMT6 cells via subcutaneous injection into the right inguinal mammary fat pad. On days 6, 10, and 15, animals were administered OxLys-SNA, OxLys-Mix, Lys-SNA, or saline (n=9 per group) via peritumoral at a dose of 5 nmol DNA and 20 μg protein. Tumor volumes were calculated by measuring the length and width with calipers and applying the formula V=L×W×W/2. Animal survival was monitored up to 100 days and animals sacrificed when tumor burden exceeded 1500 mm³. At day 60 post-inoculation, a subset of surviving OxLys-SNA animals (n=3) was re-challenged by inoculation with approximately 10⁶ EMT6 cells in the right inguinal mammary fat pad and monitored for evidence of tumor growth for an additional 40 days.

BMDC Activation. BMDCs were isolated and cultured as described above. On day 6, BMDCs were harvested and incubated (100,000 BMDCs/sample) with CpG-1826 (0.1 nmol) plus oxidized lysates, non-oxidized lysates, or saline (1 μg total protein) to induce BMDC maturation. After 48 hours incubation, cells were washed with DPBS (3×) and stained with antibodies against CD40, CD80, CD86, and MHC-II, as well as with a live/dead stain, by incubation with the appropriate antibodies for 20 min at room temperature (RT). Cells were then washed with DPBS (3×) and fixed with paraformaldehyde prior to analysis using flow cytometry. Immune cells were identified by gating for CD11b+/CD11c+ double-positive cells, followed by gating for the appropriate marker (CD40, CD80, CD86, or MHC-II).

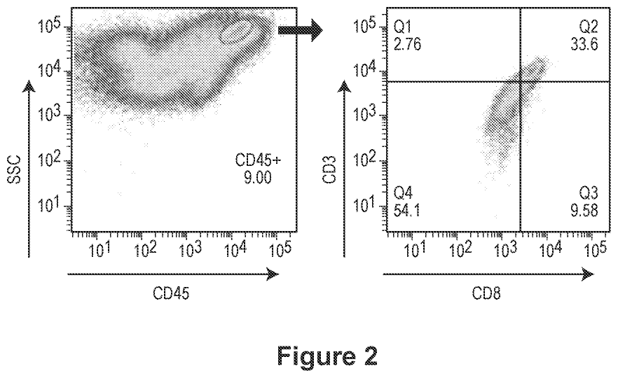

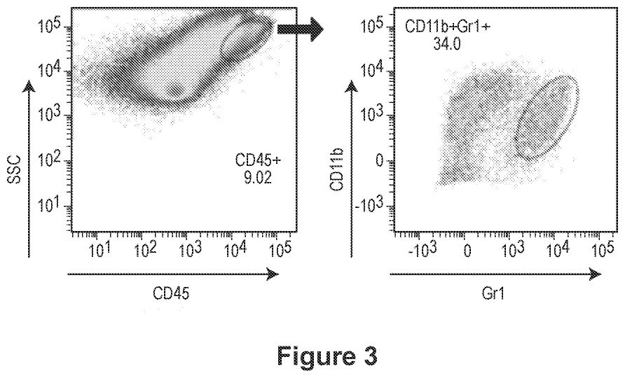

Analysis of Immune Cell Populations at EMT6 Tumor Site. Female Balb/C mice (age 8-10 weeks) were inoculated with approximately 10⁶ EMT6 cells via injection into the right inguinal mammary fat pad. At day 6 and 10 post-inoculation, animals (n=3 per group) were administered OxLys-SNA, OxLys-Mix, Lys-SNA, or saline via peritumoral injection. On day 11, animals were sacrificed and tumors harvested for immune cell population analysis. Tumors were washed with DPBS and dissociated into single cell suspensions using a cell strainer. Tumor cells were then split into two samples. One sample stained with antibodies against CD45, CD3, and CD8 to identify CD8+ cytotoxic T cells. The second sample was incubated with CD45, CD11b, and Gr1 to identify MDSCs. After incubation for 20 minutes at RT, cells were washed with DPBS (3×) and fixed with paraformaldehyde prior to analysis via flow cytometry. CD8+ T cells were identified by first gating for CD45+ cells, followed by gating for CD3+/CD8+ double-positive cells (FIG. 2). MDSCs were identified by first gating for CD45+ cells, followed by gating for CDIIb+/Gr1+ double-positive cells (FIG. 3).

### Results

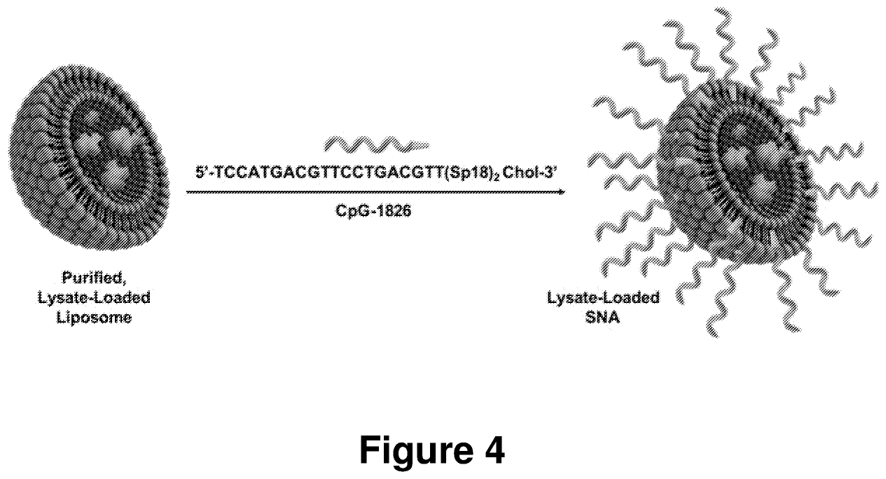

Lysates from TNBC Cell Lines can be Compartmentalized in Lys-SNAs. To assess the feasibility of using TNBC lysates as antigen sources, three murine mammary carcinoma cell lines were utilized to recapitulate the heterogeneity of TNBC (58, 59). Towards this end, Py230 was utilized, a luminal cell line (60-62), and Py8119, a basal cell line (60-62), derived from the mouse mammary tumor virus-polyoma middle tumor antigen (MMTV-PyMT) mouse model of breast cancer, which loses expression of estrogen and progesterone as it progresses (63). The EMT6 cell line was chosen as a third model, as this syngeneic line has been recently recognized as a valuable model to study immune response in TNBC (64, 65). Cells were grown to confluency in monolayer cell culture, dissociated and subjected to several freeze-thaw cycles to induce cell necrosis and rupture cell membranes, and centrifuged to remove cellular debris. The soluble protein fraction was encapsulated in approximately 70-nm liposomes prepared from 1,2-dioleoyl-sn-glycero-3-phosphocholine (DOPC). After purification to remove unencapsulated lysate, the liposomes were incubated with 3′-cholesteryl-modified CpG-1826 (FIG. 4) to generate Lys-SNAs (FIG. 1a), whose single-lamellar spherical morphologies were validated by cryogenic transmission electron microscopy (cryo-TEM, FIG. 1b). The average ratio of protein to DNA was determined to be 1.1±0.65 mg protein per μmol DNA for three independent batches of EMT6 Lys-SNAs. Analysis via gel electrophoresis (FIG. 1c) and increase in hydrodynamic diameter, as measured by dynamic light scattering (DLS, FIG. 1d) are consistent with DNA functionalization and SNA generation.

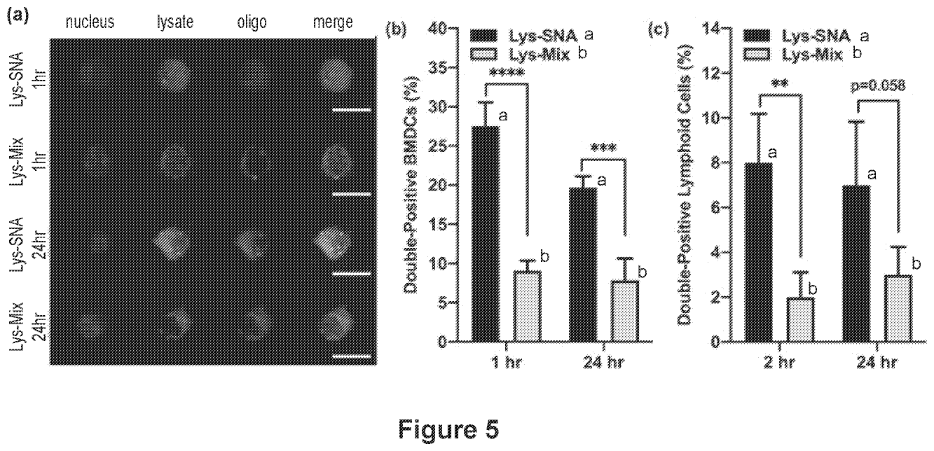

Lys-SNAs Increase Codelivery of Lysates and CpG DNA to DCs in vitro and in vivo. Codelivery of antigen and adjuvant to the same APC is vital for maximal antigen processing and presentation, as well as inducing the most potent antigen-specific immune response (66). Thus, the codelivery of lysate and DNA to APCs was investigated in vitro and in vivo. Towards this end, Lys-SNAs containing fluorophore-labeled lysate (fluorescein and Oregon Green 488) and CpG-1826 (Cy5) were synthesized. Purified lysates were incubated with an Oregon Green 488-succinimidyl ester (OR488-NHS) dye and fluorescein-5-maleimide (FITC-maleimide) dye to label both the free amines and thiols in the bulk protein solution. Following removal of any unreacted dye, FITC/OR488-labeled lysates and Cy5-modified DNA were used to generate dual fluorophore-labeled Lys-SNAs. Bone marrow-derived dendritic cells (BMDCs) were isolated from C57Bl/6 mice and incubated with either fluorophore-labeled Lys-SNAs or a mixture of fluorophore-labeled lysate and CpG-1826 (FIG. 5) at the same protein and DNA concentrations as Lys-SNAs. At set time points, cells were collected and analyzed by both confocal microscopy (FIG. 5a) and flow cytometry (FIG. 5b) to determine the number of cells positive for both FITC/OR488 and Cy5. At all time points, Lys-SNAs showed higher codelivery to immune cells in vitro than the simple mixture of lysate with CpG-1826 (Lys-Mix), showing a 3-fold and 2.5-fold enhancement in codelivery after 2 and 24 hour incubation, respectively. To evaluate codelivery of antigen and adjuvant in vivo, C57Bl/6 mice were subcutaneously administered Lys-SNA or Lys-Mix containing fluorophore-labeled lysate and CpG-1826 (n=3 per group). At 2 and 24 hours post-injection, animals were sacrificed, inguinal lymph nodes were isolated and dissociated into single-cell suspensions, and then analyzed by flow cytometry (FIG. 5c). In agreement with the in vitro data, codelivery to CD11c+ immune cells in vivo was enhanced 4-fold when lysates and adjuvant DNA were formulated as Lys-SNAs at 2 hour post-injection, with over 2.3-fold enhancement still observed at 24 hours.

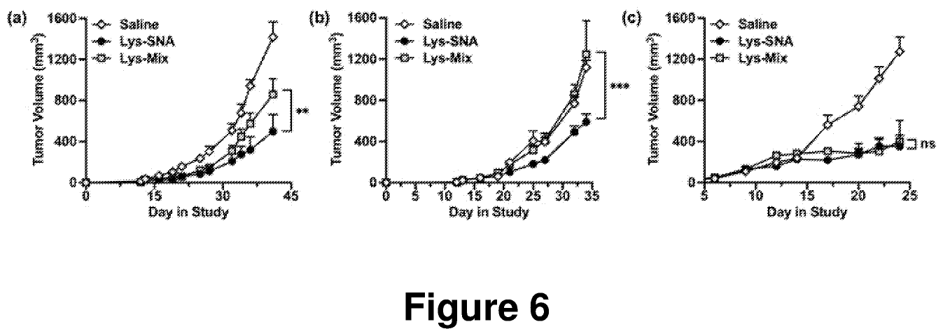

Lys-SNAs Show Antitumor Activity in Multiple TNBC Models. To assess the antitumor activity of Lys-SNAs in vivo, orthotopic syngeneic models of TNBC were established by inoculating mice with approximately 10⁶ TNBC cells (Py230, Py8119, or EMT6) in the left inguinal mammary fat pad. At days 6, 10, and 15 post-inoculation, animals were peritumorally administered Lys-SNA, Lys-Mix, or saline as a negative control at a dose of 10 nmol CpG-1826 and 20 μg lysate. In the Py230 and Py8119 models, animals administered Lys-SNAs showed a 42% and 53% reduction in tumor volume, respectively, relative to animals administered Lys-Mix at day 30 of the study (FIG. 6a-b), suggesting that packaging lysates into the core of SNAs increases their antitumor efficacy. In the EMT6 model, administration of lysate with CpG-1826 stalled tumor growth relative to saline, showing a 73% reduction in tumor growth at day 25; however, no significant difference in tumor growth was observed between animals administered Lys-SNA and Lys-Mix (FIG. 6c). Based on previous reports of using tumor lysates as antigen sources (22-24), it was hypothesized that the lysates generated in the EMT6 model were poorly immunogenic and thus leading to suboptimal T cell priming and subsequent antitumor activity. Therefore, the use of lysates derived from oxidized tumor cells was investigated in this model.

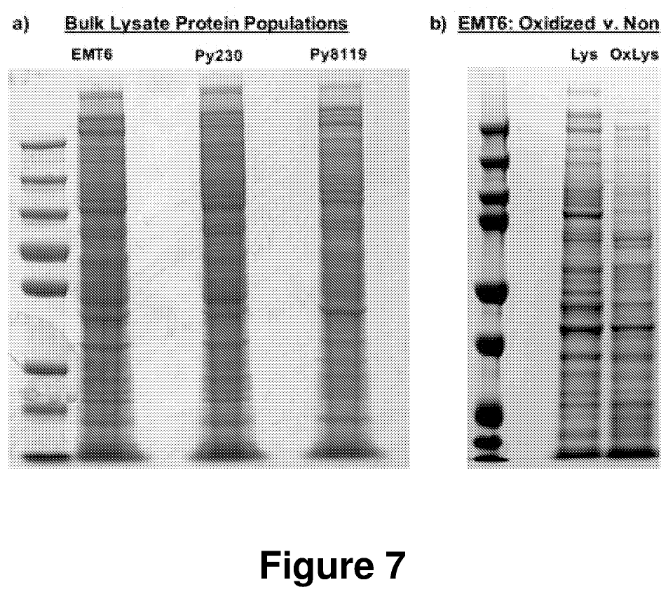

Oxidizing Tumor Cells Prior to Lysis Increases Observed Immunogenicity. Since it has been reported that oxidation increases the immunogenicity of tumor lysates, it was sought to determine whether lysates derived from oxidized tumor cells could be utilized as potent antigen sources following incorporation into SNAs. Oxidized tumor cell lysates were generated by incubating EMT6 cells in 60 μM hypochlorous acid (HOCl) for 1 hour to ensure complete cell death (67). Oxidized lysates were then prepared by subjecting the cells to several freeze-thaw cycles and centrifugation to remove cellular debris. The total amount of protein lysate collected from oxidized tumor cells (“oxidized lysate”) was similar to that of protein lysate collected from non-oxidized tumor cells (“non-oxidized lysate”); however, the bulk protein population of oxidized lysate differed from that of non-oxidized lysate, with larger protein bands appearing in the oxidized sample (FIG. 7).

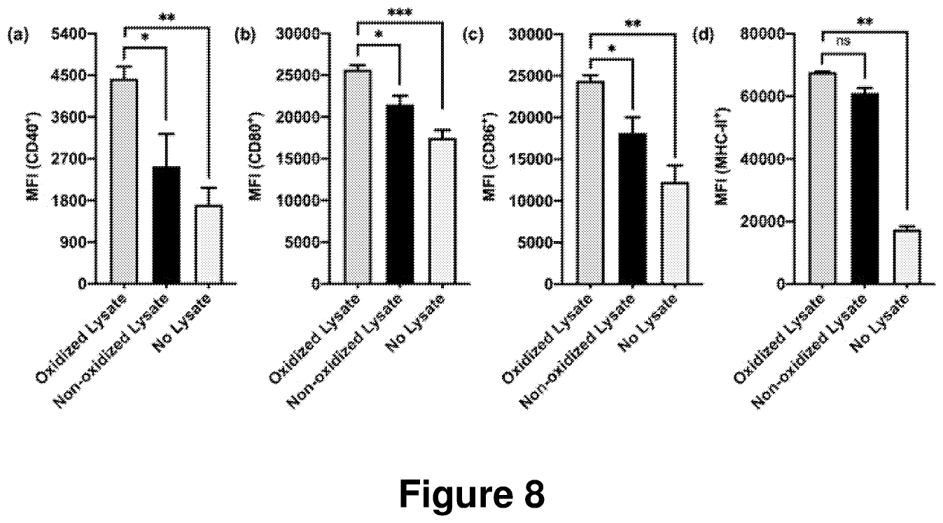

To determine whether oxidation prior to lysate generation increases the immunogenicity of the isolated lysate, BMDCs were incubated with CpG-1826 (0.1 nmol) and either non-oxidized lysate or oxidized lysate at equivalent protein concentrations (1 μg total protein). The expression of maturation markers CD40, CD80, and CD86 was significantly elevated in BMDCs incubated with oxidized lysates (FIG. 8a-c) relative to treatment with non-oxidized lysates alone, showing a 75%, 20%, and 34% increase in CD40, CD80, and CD86 marker expression over non-oxidized lysates, respectively, as well as a 160%, 47%, and 98% increase over CpG-1826 alone. Furthermore, expression of the MHC-II was elevated by 47% in BMDCs incubated with oxidized lysate as compared to those incubated with CpG-1826 alone (FIG. 8d).

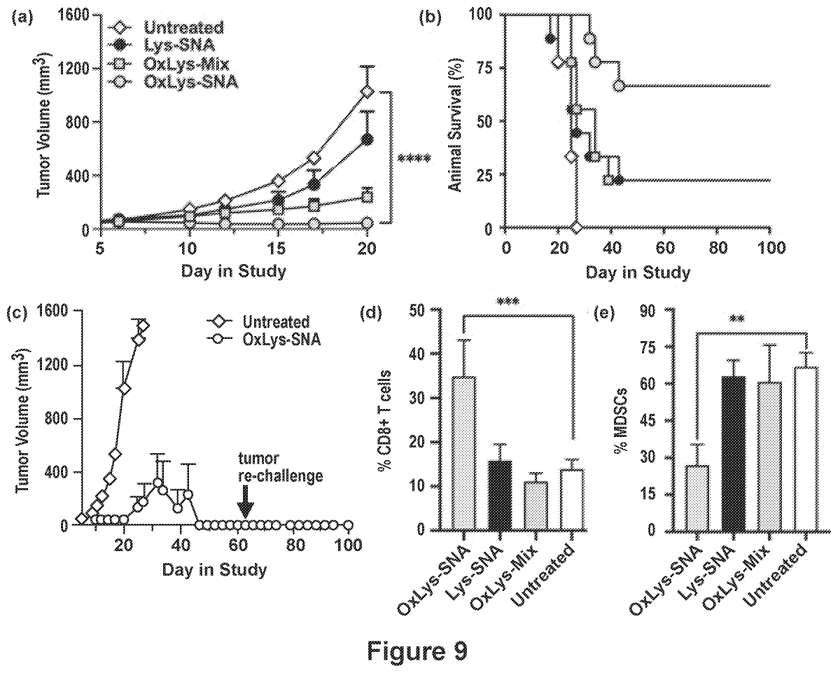

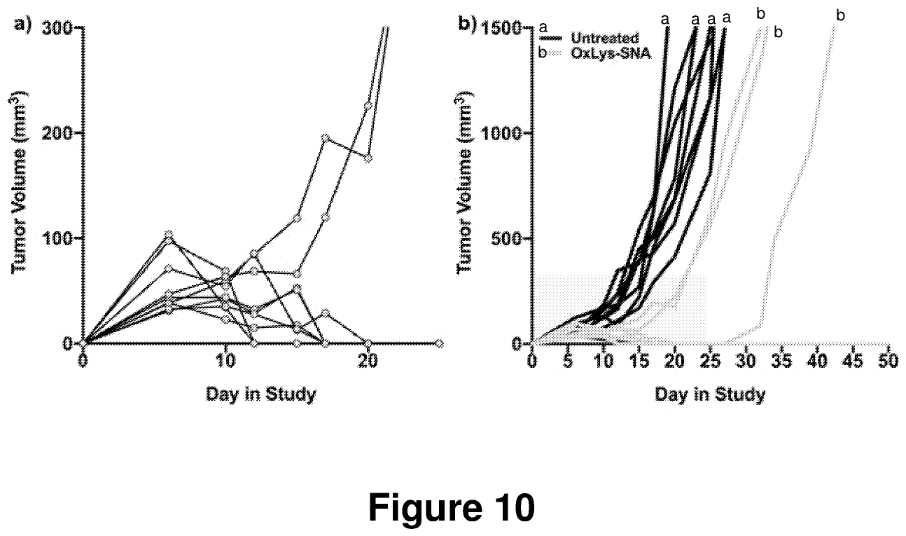

OxLys-SNAs Significantly Inhibit Tumor Growth and Extend Survival in vivo. To evaluate the function of OxLys-SNAs as cancer immunotherapeutics, the in vivo antitumor activity of OxLys-SNAs was compared to Lys-SNAs and mixtures of oxidized lysate with CpG-1826 (OxLys-Mix) in the EMT6 model of TNBC. Balb/C mice were inoculated with approximately 10⁶ tumor cells in the left inguinal mammary fat pad and at day 6 post-inoculation, treatment was initiated via peritumoral subcutaneous injection of OxLys-SNA, Lys-SNA, OxLys-Mix, (n=9 per group) at a dose of 5 nmol DNA and 20 μg protein. Saline-treated animals were used as a negative control. Injections were repeated at day 10 and 15. Tumor mass and animal survival were monitored for 100 days post-inoculation. Animals administered OxLys-SNA responded remarkably well to treatment as compared to all other treatment groups (FIG. 9a), with 7 out of 9 OxLys-SNA treated animals experiencing complete tumor remission at day 20 (FIG. 10a). Furthermore, animal survival was significantly extended when administered OxLys-SNA (FIG. 9b), with 6 out of 9 animals OxLys-SNA treated animals surviving beyond day 100 post-inoculation. Indeed, the first OxLys-SNA treated animal to succumb to tumor burden (tumor volume exceeding 1500 mm³) survives longer than all saline-treated animals (FIG. 10b). To assess the capability of OxLys-SNAs to impart memory immune response, a small cohort of animals administered OxLys-SNAs were re-challenged by implanting approximately 10⁶ EMT6 cells in the inguinal mammary fat pad on day 60 following initial tumor implantation (FIG. 9c). All animals (n=3) remained tumor free following re-challenge with EMT6 cells, indicating that vaccination with OxLys-SNAs not only eradicates existing tumors but also prevents new tumors from forming.

OxLys-SNAs Alter the Immune Cell Population Within the Tumor Microenvironment. To determine the effect of OxLys-SNA administration on the immune cell population at the tumor site, Balb/c mice were inoculated with EMT6 cells in the left inguinal mammary fat pad. At days 6 and 10 post-inoculation, animals were administered OxLys-SNA, Lys-SNA, OxLys-Mix, or saline (n=3 per group) via peritumoral subcutaneous injection. On day 11, animals were sacrificed. Tumors were dissociated into single cell suspensions and split into two fractions. One cell fraction was incubated with antibodies against CD45, CD3, and CD8 to identify CD8+ (cytotoxic) T cells present in the tumor microenvironment. The second cell fraction was incubated with CD45, CD11b, and Gr1 to identify myeloid derived suppressor cells (MDSCs) present in the tumor microenvironment. Following antibody incubation, cells were fixed and analyzed via flow cytometry. Excitingly, the population of CD8+ T cells at the tumor site was significantly elevated in animals administered OxLys-SNAs (FIG. 9d) relative to all other treatment groups, showing a 2.3-fold increase relative to saline-treated controls. Concurrently, the population of MDSCs in animals administered OxLys-SNAs (FIG. 9e) was decreased 2.5-fold relative to saline-treated controls.

### Discussion

Encapsulating tumor cell lysates into the core of liposomal SNAs increases the codelivery of lysate and adjuvant DNA to immune cells relative to simple mixtures, both in vitro and in vivo. This highlights the importance of structural arrangement in the design of immunotherapeutics, as the maximal immune response is achieved when both adjuvant and antigen are delivered to the same target cell (66). Indeed, formulation of lysate and CpG-1826 into SNAs leads to higher codelivery of both immunomodulatory components to the same CD11c+ cells in the draining lymph node in vivo at 2 hour post-injection, as compared to a simple mixture of linear CpG-1826 and bare liposomes containing lysate. Importantly, the percent of CD11c+ cells staining positive for both Cy5-labeled DNA and fluorophore-labeled lysate is maintained up to 24 hours after in vivo delivery with Lys-SNAs, whereas the percentage of double positive cells increased when treated with the simple mixture, but not to the level of Lys-SNAs.

Furthermore, generating lysates from cells that have been subjected to oxidative stress with HOCl to induce cell death results in protein populations of higher molecular weight than those isolated from cells that have not been subjected to oxidative stress. This finding confirms that cellular oxidation prior to lysate generation changes the available antigen pool, which is in agreement with published studies on the use of oxidized lysates in DC vaccines (20, 68). When incubated with BMDCs in vitro, oxidized lysates enhance DC maturation over both non-oxidized lysates and CpG-1826. This finding suggested that inducing cell death via oxidation leads to lysate populations that have elevated adjuvant behavior as well as antigenic behavior, as DC maturation is primarily dictated by activation with adjuvants (69).

In the EMT6 model of TNBC, OxLys-SNAs show significant antitumor activity, with 6 out of 9 animals experiencing complete tumor remission by day 20. Intriguingly, the first animal administered OxLys-SNA does not succumb to tumor burden (day 32 post-inoculation) until 5 days after the last saline-treated animal (day 27 post-inoculation). Thus, OxLys-SNAs act as potent immunotherapeutics, with superior performance to both Lys-SNAs and OxLys-Mix. Excitingly, animals who received OxLys-SNAs resisted re-challenge with EMT6 cells at 60 days post-inoculation, indicating that this treatment regime has the potential to offer protective immunity. Moreover, OxLys-SNAs significantly increase the populations of cytotoxic CD8+ T cells and simultaneously decrease the population of MDSCs in the TME, relative to all other treatment groups. High levels of cytotoxic T cells in the microenvironment of breast tumors have been shown to be correlated with positive antitumor effects (70, 71), while high levels of MDSCs promote immune evasion (72). Therefore, this finding provides insight on the mechanism responsible for the observed antitumor efficacy of OxLys-SNAs.

This work is important because it describes a new class of potent vaccines based upon the compartmentalization of antigens in the form of oxidized lysate within a novel nanotherapeutic. These structures, in three TNBC tumor models, showed very promising activity with respect to codelivery of lysate and adjuvant and DNA, antitumor efficacy, extended animal survival, and alteration of the immune cell population within the TME. Taken together, these results showed that the method in which tumor cell lysates are generated, as well as the way adjuvant and antigen(s) are packaged and delivered to the immune system, has a profound impact on the resulting antitumor efficacy. Therefore, these results have important implications in the development of personalized immunotherapies for TNBC and other cancers.

### Conclusion

In mouse models of triple negative breast cancer, it is shown herein that the oxidation of tumor cells prior to lysate generation, coupled with their compartmentalization in the core of liposomal spherical nucleic acids (SNAs) comprised of adjuvant DNA, yields a powerful immunotherapeutic that significantly inhibits tumor growth, dramatically extends survival, and promotes a tumoricidal immune cell population within the tumor microenvironment. Specifically, this work demonstrated the importance of properly packaging and presenting the adjuvant and antigens such that biodistribution, dendritic cell activation, and therapeutic efficacy can be controlled.

### Example 2

This example describes results of additional experiments designed to test the antitumor activity of liposomal SNAs in the Py8119 model of TNBC. More specifically, the experiments were designed to test the ability of liposomal SNAs comprising DPPC and containing tumor lysates with liposomal SNAs (“L-SNAs”) comprising DOPC and containing tumor lysates.

### Materials and Methods

Generation of Lysates from Py8119 Cells. Py8119 cells were obtained from ATCC and grown in F-12K medium supplemented with 5% FBS and 1% penicillin-streptomycin. To prepare lysates, cells were dissociated from the culture plate using trypsin, washed, collected, and resuspended at 1×10⁶ cells/mL in Dulbecco's phosphate buffered saline (DPBS). The suspension was then subjected to four freeze-thaw cycles, alternating between submersion in liquid nitrogen and a 37° C. water bath. Cellular debris was removed via centrifugation at 10,000 RCF for 10 minutes and the supernatant was collected as the lysate. Total protein concentration was measured using a Bradford assay, with albumin as the protein standard (Pierce, ThermoFisher Scientific). To prepare oxidized lysates, Py8119 cells were grown to confluence in monolayer cell culture. Cells were washed with DPBS three times, then incubated for 1 hour at 37° C. with 60 μM hypochlorous acid (HOCl) in DPBS. Dissociated cells were collected and washed with DPBS to remove any unreacted HOCl, centrifuged at 500 RCF for 5 minutes, and resuspended in DPBS at a density of 1×10⁷ cells/mL. Cells were subjected to four freeze-thaw cycles and centrifuged as described above. The soluble fraction was collected as the oxidized lysate and protein concentration analyzed using the Bradford assay.

Synthesis of Lysate-Loaded L-SNAs. Lysates were encapsulated into liposomes using the thin-film rehydration method. Briefly, lysates (both normal and oxidized) were suspended in DPBS at 1.0 mg/mL with respect to protein concentration and 1 mL of each solution was added to vials containing 10 mg of DOPC or 10 mg of DPPC. Liposomes were formed through several freeze-thaw/sonication cycles, using liquid nitrogen and sonication in a 37° C. water bath, until the hydrodynamic diameter by DLS was below 100 nm (Table 2) using a Malvern Zetasizer. Tangential flow filtration (TFF) with a pore size of 100 kDa (Spectrum) was used to remove any unencapsulated lysate from the liposome solutions. Protein encapsulated within the liposomes was quantified using the Bradford assay, following liposome disruption using 1% sodium dodecyl sulfate. To form SNAs, cholesterol-terminated oligonucleotides (5′-TCCATGACGTTCCTGACGTT(Spacer-18 (hexaethyleneglycol))₂Cholesterol-3′ (SEQ ID NO: 2)) were embedded into the outer membrane of the liposomes by mixing 20 μM oligonucleotides with the solutions of lysate-loaded liposomes at T>TC overnight. The oligonucleotide concentration was determined using the absorption at 260 nm with UV-vis spectroscopy (Cary). The resulting SNAs were then concentrated to 20 μM by DNA using centrifugation filter units (Millipore) with a 3 KD molecular weight cutoff, which also removed any unbound DNA and analyzed with DLS to confirm successful DNA functionalization (Table 2).

TABLE 2

| | | | SNA | | SNA | |
|---|---|---|---|---|---|---|
| Lipid | Liposome | | 50 strands/particle | | 100 strands/particle | |
| Core | D (nm) | PDI | D (nm) | PDI | D (nm) | PDI |
| DOPC | 89.9 | 0.19 | 110.0 | 0.12 | 127.5 | 0.24 |
| DMPC | 81.8 | 0.15 | 115.6 | 0.22 | 120.3 | 0.23 |
| DPPC | 83.6 | 0.06 | 129.1 | 0.027 | 127.7 | 0.01 |
| DSPC | 94.3 | 0.05 | 125.7 | 0.19 | 129.8 | 0.17 |

Antitumor Efficacy in the Py8119 Model. Female mice (Balb/C) were inoculated with 1×10⁶ Py8119 cells in the right inguinal mammary fat pad via subcutaneous injection. On days 6, 10, and 15 post-inoculation, animals were administered DOPC-SNAs, DPPC-SNAs, DOPC-Lys-SNAs, DPPC-Lys-SNAs, DOPC-OxLys-SNAs, DPPC-OxLys-SNAs, or saline (n=4 per group) via peritumoral subcutaneous injection (5 nmol DNA in 100 μL). Tumor volumes were calculated by measuring the length and width with calipers and applying the formula V=L/2×W×W. Relative tumor volume was calculated using the formula Vᵣₑₗ=V/V₀, where V=volume on the day of measurement and V₀=tumor volume on the day of the first injection. The study was halted at day 24 due to tumor ulceration in untreated animals.

### Results

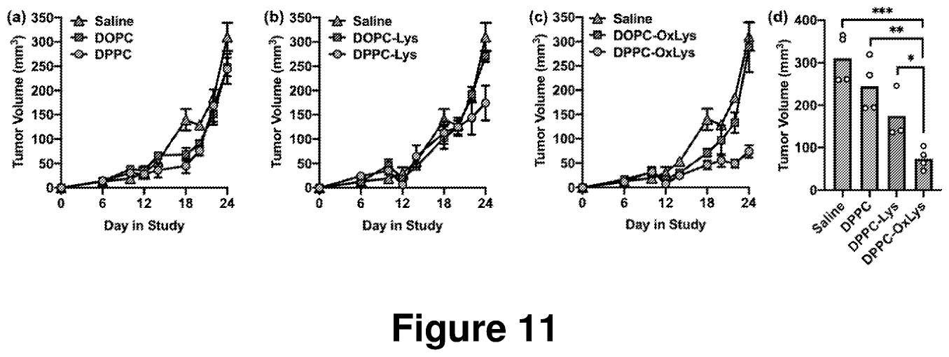

FIG. 11a shows results of experiments in which DPPC-SNAs and DOPC-SNAs were administered as “adjuvant only” immunotherapeutics. Because no TAAs have been identified for TNBC, lysates from Py8119 cells were generated and then utilized as antigen sources. These lysates were encapsulated into L-SNAs comprised of DOPC (DOPC-Lys-SNAs) or DPPC (DPPC-Lys-SNAs). The antitumor efficacy of both constructs was compared in vivo following L-SNA administration at days 6, 10, and 15 at a dose of 5 nmol of CpG-1826 and 10 μg of lysate. Results showed an approximate 60% reduction in tumor growth when DPPC-Lys-SNAs were administered as compared to animals administered saline, whereas there was no difference in tumor growth in animals administered DOPC-Lys-SNAs (FIG. 11b), again showing the dependence of antitumor efficacy on L-SNA stability.

As shown in Example 1, incorporating oxidized lysates into the core of L-SNAs leads to dramatic increases in antitumor efficacy in mouse models of TNBC. Therefore, to determine whether the effects of tumor cell oxidation and L-SNA stability were additive, L-SNAs containing lysates from oxidized Py8119 cells were synthesized using DOPC (DOPC-OxLys-SNAs) and DPPC (DPPC-OxLys-SNAs). DPPC-OxLys-SNAs significantly suppressed tumor growth over the duration of the study (FIG. 11c), while DOPC-OxLys-SNAs were less effective at these doses of DNA and lysate. Collectively, these data indicate that the effects of lysate preparation method and L-SNA stability are synergistic, a trend which became clearer when the study endpoint for all DPPC-based L-SNAs is compared (FIG. 11d). The inclusion of lysates into the L-SNA (DPPC-Lys-SNAs) was shown to be effective, and maximum antitumor efficacy was observed when lysates from oxidized cells were used as the antigen sources (DPPC-OxLys-SNAs), revealing the importance of both antigen processing method and liposome stability in these constructs.

### CONCLUSIONS

In the Py8119 mouse model, lysates encapsulated in L-SNAs synthesized with DPPC were more effective than their DOPC analogues at stalling tumor growth; a trend that became more pronounced when incorporating lysates from oxidized Py8119 cells into the L-SNA scaffold, revealing synergy between lysate incorporation method and L-SNA stability. Note, however, that the DOPC SNAs used in Example 2 were administered in a different model of TNBC than Example 1 (EMT6 for Example 1, 4T1 and Py8119 for Example 2). Further, the dose of adjuvant DNA administered in Example 2 was 50% of that used in Example 1.

### REFERENCES

1. S. A. Ali, V. Shi, I. Maric, M. Wang, D. F. Stroncek et al., T Cells Expressing an Anti-B-Cell Maturation Antigen Chimeric Antigen Receptor Cause Remissions of Multiple Myeloma. Blood 128, 1688-1700 (2016).
2. S. L. Maude, T. W. Laetsch, J. Buechner, S. Rives, M. Boyer et al., Tisagenlecleucel in Children and Young Adults with B-Cell Lymphoblastic Leukemia. N. Engl. J. Med. 378, 439-448 (2018).
3. S. S. Neelapu, F. L. Locke, N. L. Bartlett, L. J. Lekakis, D. B. Miklos et al., Axicabtagene Ciloleucel CAR T-Cell Therapy in Refractory Large B-Cell Lymphoma. N. Engl. J. Med. 377, 2531-2544 (2017).
4. J. H. Park, I. Riviere, M. Gonen, X. Wang, B. Senechal et al., Long-Term Follow-up of CD19 CAR Therapy in Acute Lymphoblastic Leukemia. N. Engl. J. Med. 378, 449-459 (2018).
5. S. J. Schuster, J. Svoboda, E. A. Chong, S. D. Nasta, A. R. Mato et al., Chimeric Antigen Receptor T Cells in Refractory B-Cell Lymphomas. N. Engl. J. Med. 377, 2545-2554 (2017)
6. M. John M. Timmerman, M. Ronald Levy, Dendritic Cell Vaccines for Cancer Immunotherapy. Annu. Rev. Med. 50, 507-529 (1999).
7. C. E. Brown, C. L. Mackall, CAR T cell therapy: inroads to response and resistance. Nat. Rev. Immunol. 19, 73-74 (2019).
8. R. Kuai, L. J. Ochyl, K. S. Bahjat, A. Schwendeman, J. J. Moon, Designer Vaccine Nanodiscs for Personalized Cancer Immunotherapy. Nat. Mater. 16, 489 (2016).
9. J. Fang, B. Hu, S. Li, C. Zhang, Y. Liu et al., A Multi-Antigen Vaccine in Combination with an Immunotoxin Targeting Tumor-Associated Fibroblast for Treating Murine Melanoma. Mol. Ther.—Oncolytics 3, 16007-16007 (2016).
10. C. L. Slingluff, Jr., The Present and Future of Peptide Vaccines for Cancer: Single or Multiple, Long or Short, Alone or in Combination? Cancer J. 17, 343-350 (2011).
11. F. R. Balkwill, M. Capasso, T. Hagemann, The Tumor Microenvironment at a Glance. J. Cell Sci. 125, 5591-5596 (2012).
12. M. Kawahara, H. Takaku, A Tumor Lysate is an Effective Vaccine Antigen for the Stimulation of CD4(+) T-Cell Function and Subsequent Induction of Antitumor Immunity Mediated by CD8(+) T Cells. Cancer Biol. Ther. 16, 1616-1625 (2015).
13. F. E. Gonzalez, A. Gleisner, F. Falcon-Beas, F. Osorio, M. N. Lopez et al., Tumor Cell Lysates as Immunogenic Sources for Cancer Vaccine Design. Hum. Vaccines Immunother. 10, 3261-3269 (2014).
14. J. S. Moyer, G. Maine, J. J. Mule, Early Vaccination with Tumor-Lysate-Pulsed Dendritic Cells after Allogeneic Bone Marrow Transplantation has Antitumor Effects. Biol. Blood Marrow Transplant. 12, 1010-1019 (2006).
15. S. J. Win, D. G. McMillan, F. Errington-Mais, V. K. Ward, S. L. Young et al., Secretomes from Metastatic Breast Cancer Cells, Enriched for a Prognostically Unfavorable LCN2 Axis, Induce Anti-Inflammatory MSC Actions and a Tumor-Supportive Premetastatic Lung. Br. J. Cancer 106, 92-98 (2012).
16. R. C. Fields, K. Shimizu, J. J. Mule, Murine Dendritic Cells Pulsed with Whole Tumor Lysates Mediate Potent Antitumor Immune Responses in vitro and in vivo. Proc. Natl. Acad. Sci. U.S.A. 95, 9482-9487 (1998).
17. J. Kim, D. J. Mooney, In Vivo Modulation of Dendritic Cells by Engineered Materials: Towards New Cancer Vaccines. Nano Today 6, 466-477 (2011).
18. L. C. Chiang, G. Coukos, E. L. Kandalaft, Whole Tumor Antigen Vaccines: Where Are We? Vaccines 3 (2015).
19. C. L.-L. Chiang, F. Benencia, G. Coukos, Whole tumor antigen vaccines. Seminars in Immunology 22, 132-143 (2010).
20. M. L. Grant, N. Shields, S. Neumann, K. Kramer, A. Bonato et al., Combining dendritic cells and B cells for presentation of oxidised tumour antigens to CD8(+) T cells. Clinical & translational immunology 6, e149-e149 (2017).
21. C. L. Chiang, L. E. Kandalaft, J. Tanyi, A. R. Hagemann, G. T. Motz et al., A dendritic cell vaccine pulsed with autologous hypochlorous acid-oxidized ovarian cancer lysate primes effective broad antitumor immunity: from bench to bedside. Clin. Cancer Res. 19, 4801-4815 (2013).
22. J. Marcinkiewicz, Neutrophil Chloramines: Missing Links Between Innate and Acquired Immunity. Immunol. Today 18, 577-580 (1997).
23. J. Marcinkiewicz, B. M. Chain, E. Olszowska, S. Olszowski, J. M. Zgliczyński, Enhancement of Immunogenic Properties of Ovalbumin as a Result of its Chlorination. Int. J. Biochem. 23, 1393-1395 (1991).
24. M. E. D. Allison, D. T. Fearon, Enhanced Immunogenicity of Aldehyde-Bearing Antigens: A Possible Link Between Innate and Adaptive Immunity. Eur. J. Immunol. 30, 2881-2887 (2000).
25. P. G. Coulie, B. J. Van den Eynde, P. van der Bruggen, T. Boon, Tumour Antigens Recognized by T Lymphocytes: At the Core of Cancer Immunotherapy. Nat. Rev. Cancer 14, 135-146 (2014).
26. S. Frey, A. Castro, A. Arsiwala, R. S. Kane, Bionanotechnology for Vaccine Design. Curr. Opin. Biotechnol. 52, 80-88 (2018).
27. D. J. Irvine, M. C. Hanson, K. Rakhra, T. Tokatlian, Synthetic Nanoparticles for Vaccines and Immunotherapy. Chem. Rev. (Washington, D.C., U.S.) 115, 11109-11146 (2015).
28. J. A. Kemp, M. S. Shim, C. Y. Heo, Y. J. Kwon, “Combo” Nanomedicine: Co-Delivery of Multi-Modal Therapeutics for Efficient, Targeted, and Safe Cancer Therapy. Adv. Drug Delivery Rev. 98, 3-18 (2016).
29. C. A. Mirkin, R. L. Letsinger, R. C. Mucic, J. J. Storhoff, A DNA-based method for rationally assembling nanoparticles into macroscopic materials. Nature 382, 607 (1996).
30. J. I. Cutler, E. Auyeung, C. A. Mirkin, Spherical nucleic acids. J. Am. Chem. Soc. 134, 1376-1391 (2012).
31. D. S. Seferos, A. E. Prigodich, D. A. Giljohann, P. C. Patel, C. A. Mirkin, Polyvalent DNA Nanoparticle Conjugates Stabilize Nucleic Acids. Nano Lett. 9, 308-311 (2009).
32. A. K. R. Lytton-Jean, J. M. Gibbs-Davis, H. Long, G. C. Schatz, C. A. Mirkin et al., Highly Cooperative Behavior of Peptide Nucleic Acid-Linked DNA-Modified Gold-Nanoparticle and Comb-Polymer Aggregates. Adv. Mater. 21, 706-709 (2009).
33. J.-S. Lee, A. K. R. Lytton-Jean, S. J. Hurst, C. A. Mirkin, Silver Nanoparticle-Oligonucleotide Conjugates Based on DNA with Triple Cyclic Disulfide Moieties. Nano Lett. 7, 2112-2115 (2007).
34. G. P. Mitchell, C. A. Mirkin, R. L. Letsinger, Programmed Assembly of DNA Functionalized Quantum Dots. J. Am. Chem. Soc. 121, 8122-8123 (1999).
35. J. I. Cutler, D. Zheng, X. Xu, D. A. Giljohann, C. A. Mirkin, Polyvalent Oligonucleotide Iron Oxide Nanoparticle “Click” Conjugates. Nano Lett. 10, 1477-1480 (2010).
36. K. L. Young, A. W. Scott, L. Hao, S. E. Mirkin, G. Liu et al., Hollow Spherical Nucleic Acids for Intracellular Gene Regulation Based Upon Biocompatible Silica Shells. Nano Lett. 12, 3867-3871 (2012).
37. R. J. Banga, N. Chernyak, S. P. Narayan, S. T. Nguyen, C. A. Mirkin, Liposomal Spherical Nucleic Acids. J. Am. Chem. Soc. 136, 9866-9869 (2014).
38. A. J. Sprangers, L. Hao, R. J. Banga, C. A. Mirkin, Liposomal Spherical Nucleic Acids for Regulating Long Noncoding RNAs in the Nucleus. Small 13, 1602753 (2016).
39. B. Meckes, R. J. Banga, S. T. Nguyen, C. A. Mirkin, Enhancing the Stability and Immunomodulatory Activity of Liposomal Spherical Nucleic Acids through Lipid-Tail DNA Modifications. Small 14, 1702909 (2017).
40. A. F. Radovic-Moreno, N. Chernyak, C. C. Mader, S. Nallagatla, R. S. Kang et al., Immunomodulatory Spherical Nucleic Acids. Proc. Natl. Acad. Sci. U.S.A. 112, 3892-3897 (2015).
41. K. Skakuj, S. Wang, L. Qin, A. Lee, B. Zhang et al., Conjugation Chemistry-Dependent T-Cell Activation with Spherical Nucleic Acids. Journal of the American Chemical Society 140, 1227-1230 (2018).
42. Z. Li, Y. Zhang, P. Fullhart, C. A. Mirkin, Reversible and Chemically Programmable Micelle Assembly with DNA Block-Copolymer Amphiphiles. Nano Lett. 4, 1055-1058 (2004).
43. C. M. Calabrese, T. J. Merkel, W. E. Briley, P. S. Randeria, S. P. Narayan et al., Biocompatible infinite-coordination-polymer nanoparticle-nucleic-acid conjugates for antisense gene regulation. Angew. Chem., Int. Ed. 54, 476-480 (2014).
44. S. Zhu, H. Xing, P. Gordiichuk, J. Park, C. A. Mirkin, PLGA Spherical Nucleic Acids. Adv. Mater. 30, 1707113 (2018).
45. J. D. Brodin, A. J. Sprangers, J. R. McMillan, C. A. Mirkin, DNA-Mediated Cellular Delivery of Functional Enzymes. J. Am. Chem. Soc. 137, 14838-14841 (2015).
46. U. Bulbake, S. Doppalapudi, N. Kommineni, W. Khan, Liposomal Formulations in Clinical Use: An Updated Review. Pharmaceutics 9, 12 (2017).
47. A. F. Radovic-Moreno, N. Chernyak, C. C. Mader, S. Nallagatla, R. S. Kang et al., Immunomodulatory spherical nucleic acids. Proceedings of the National Academy of Sciences 112, 3892-3897 (2015).
48. C. Guan, N. Chernyak, D. Dominguez, L. Cole, B. Zhang et al., RNA-Based Immunostimulatory Liposomal Spherical Nucleic Acids as Potent TLR7/8 Modulators. Small 10.1002/smII.201803284, e1803284 (2018).
49. Z. N. Huang, L. E. Cole, C. E. Callmann, S. Wang, C. A. Mirkin, Sequence Multiplicity within Spherical Nucleic Acids. ACS Nano 14, 1084-1092 (2020).
50. J. Vollmer, R. Weeratna, P. Payette, M. Jurk, C. Schetter et al., Characterization of Three CpG oligodeoxynucleotide Classes with Distinct Immunostimulatory Activities. Eur. J. Immunol. 34, 251-262 (2004).
51. Y. Kumagai, O. Takeuchi, S. Akira, TLR9 as a Key Receptor for the Recognition of DNA. Adv. Drug Delivery Rev. 60, 795-804 (2008).
52. B. P. Gross, A. Wongrakpanich, M. B. Francis, A. K. Salem, L. A. Norian, A Therapeutic Microparticle-Based Tumor Lysate Vaccine Reduces Spontaneous Metastases in Murine Breast Cancer. AAPS J. 16, 1194-1203 (2014).
53. Z. Li, Y. Qiu, W. Lu, Y. Jiang, J. Wang, Immunotherapeutic interventions of Triple Negative Breast Cancer. Journal of Translational Medicine 16, 147 (2018).
54. J. Stagg, B. Allard, Immunotherapeutic Approaches in Triple-Negative Breast Cancer: Latest Research and Clinical Prospects. Ther. Adv. Med. Oncol. 5, 169-181 (2013).
55. C. Anders, L. A. Carey, Understanding and Treating Triple-Negative Breast Cancer. Oncology 22, 1233-1243 (2008).
56. F. Andre, C. C. Zielinski, Optimal Strategies for the Treatment of Metastatic Triple-Negative Breast Cancer with Currently Approved Agents. Ann. Oncol. 23 Suppl 6, vi46-51 (2012).
57. C. K. Anders, L. A. Carey, Biology, Metastatic Patterns, and Treatment of Patients with Triple-Negative Breast Cancer. Clin. Breast Cancer 9 Suppl 2, S73-S81 (2009).
58. M. Fedele, L. Cerchia, G. Chiappetta, The Epithelial-to-Mesenchymal Transition in Breast Cancer: Focus on Basal-Like Carcinomas. Cancers 9, 134 (2017).
59. Y. Su, N. R. Hopfinger, T. D. Nguyen, T. J. Pogash, J. Santucci-Pereira et al., Epigenetic Reprogramming of Epithelial Mesenchymal Transition in Triple Negative Breast Cancer Cells with DNA Methyltransferase and Histone Deacetylase Inhibitors. J. Exp. Clin. Cancer Res. 37, 314 (2018).
60. K. J. Meade, F. Sanchez, A. Aguayo, N. Nadales, S. G. Hamalian et al., Secretomes from metastatic breast cancer cells, enriched for a prognostically unfavorable LCN2 axis, induce anti-inflammatory MSC actions and a tumor-supportive premetastatic lung. Oncotarget 10, 3027-3039 (2019).
61. L. G. Ellies, C. Kuo, PyVmT Luminal and EMT Cell Lines Exhibit Characteristic Patterns of Orthotopic and Tail Vein Metastasis. Cancer Research 73, C91-C91 (2013).
62. T. Biswas, J. Yang, L. Zhao, L. Sun, Abstract P2-04-07: Modeling orthotopic and metastatic progression of mammary tumors to evaluate the efficacy of TGF-β inhibitors in a pre-clinical setting. Cancer Research 72, P2-04-07-P02-04-07 (2012).
63. J. L. Christenson, K. T. Butterfield, N. S. Spoelstra, J. D. Norris, J. S. Josan et al., MMTV-PyMT and Derived Met-1 Mouse Mammary Tumor Cells as Models for Studying the Role of the Androgen Receptor in Triple-Negative Breast Cancer Progression. Horm. Cancer 8, 69-77 (2017).
64. M. Ouzounova, E. Lee, R. Piranlioglu, A. El Andaloussi, R. Kolhe et al., Monocytic and Granulocytic Myeloid Derived Suppressor Cells Differentially Regulate Spatiotemporal Tumour Plasticity During Metastatic Cascade. Nat. Commun. 8, 14979-14979 (2017).
65. S. Barnes, “EMT-6 Syngeneic Breast Tumor Model—A Powerful Tool for Immuno-Oncology Studies.” Available at https://www.mibioresearch.com/knowledge-center/emt-6-syngeneic-breast-tumor-model-a-powerful-tool-for-immuno-oncology-studies/. Accessed 2 Mar. 2020.
66. Y. Krishnamachari, A. K. Salem, Innovative Strategies for Co-Delivering Antigens and CpG Oligonucleotides. Adv. Drug Delivery Rev. 61, 205-217 (2009).
67. Z. M. Prokopowicz, F. Arce, R. Biedron, C. L.-L. Chiang, M. Ciszek et al., Hypochlorous Acid: A Natural Adjuvant That Facilitates Antigen Processing, Cross-Priming, and the Induction of Adaptive Immunity. J. Immunol. 184, 824-835 (2010).
68. C. L. Chiang, L. E. Kandalaft, J. Tanyi, A. R. Hagemann, G. T. Motz et al., A dendritic cell vaccine pulsed with autologous hypochlorous acid-oxidized ovarian cancer lysate primes effective broad antitumor immunity: from bench to bedside. Clinical cancer research: an official journal of the American Association for Cancer Research 19, 4801-4815 (2013).
69. N. I. Ho, L. G. M. Huis In't Veld, T. K. Raaijmakers, G. J. Adema, Adjuvants Enhancing Cross-Presentation by Dendritic Cells: The Key to More Effective Vaccines? Front. Immunol. 9, 2874-2874 (2018).
70. N. R. Maimela, S. Liu, Y. Zhang, Fates of CD8+ T cells in Tumor Microenvironment. Comput. Struct. Biotechnol. J. 17, 1-13 (2018).
71. P. S. Kim, R. Ahmed, Features of Responding T cells in Cancer and Chronic Infection. Curr. Opin. Immunol. 22, 223-230 (2010).
72. A. M. Lesokhin, T. Merghoub, J. D. Wolchok, Myeloid-Derived Suppressor Cells and the Efficacy of CD8(+) T-cell Immunotherapy. OncoImmunology 2, e22764-e22764 (2013). S. Wang, L. Qin, G. Yamankurt, K. Skakuj, Z. Huang et al., Rational Vaccinology with Spherical Nucleic Acids. Proc. Natl. Acad. Sci. U.S.A. 116, 10473 (2019).
73. Callmann, C. E., Cole, Lisa E., Kusmierz, Caroline D., Huang, Ziyin, Horiuchi, Dai, Mirkin, Chad A., Tumor cell lysate-loaded immunostimulatory spherical nucleic acids as therapeutics for triple-negative breast cancer. Proc. Natl. Acad. Sci. U.S.A. 2020

What is claimed is:

1. A nanoparticle having a substantially spherical geometry comprising an oligonucleotide conjugated thereto, wherein the oligonucleotide is a Toll-Like Receptor (TLR) agonist, and wherein an oxidized tumor cell lysate is encapsulated within the nanoparticle.

2. The nanoparticle of claim 1, wherein the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLR5) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof.

3. The nanoparticle of claim 1 or claim 2, wherein the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof.

4. The nanoparticle of claim 2 or claim 3, wherein the TLR9 agonist is 5′-TCCATGACGTTCCTGACGTT-3′ (SEQ ID NO: 1).

5. The nanoparticle of any one of claims 1-4, wherein the nanoparticle is a poly(lactic-co-glycolic acid) (PLGA), poly(acrylate), or poly(methacrylate) nanoparticle.

6. The nanoparticle of claim 2 or claim 3, wherein the TLR9 agonist is 5′-TCCATGACGTTCCTGACGTT(Spacer-18 (hexaethyleneglycol))₂Cholesterol-3′ (SEQ ID NO: 2).

7. The nanoparticle of any one of claim 1-4 or 6, wherein the oligonucleotide comprises a lipophilic group.

8. The nanoparticle of claim 7, wherein the lipophilic group comprises tocopherol or cholesterol.

9. The nanoparticle of claim 8, wherein the cholesterol is cholesteryl-triethyleneglycol (cholesteryl-TEG).

10. The nanoparticle of claim 8 wherein tocopherol is a tocopherol derivative, alpha-tocopherol, beta-tocopherol, gamma-tocopherol, or delta-tocopherol.

11. The nanoparticle of any one of claim 1-4 or 6-10, wherein the nanoparticle comprises a plurality of lipid groups.

12. The nanoparticle of claim 11, wherein at least one lipid group is of the phosphatidylcholine, phosphatidylglycerol, or phosphatidylethanolamine family of lipids.

13. The nanoparticle of claim 11, wherein at least one lipid group is 1,2-dioleoyl-sn-glycero-3-phosphocholine (DOPC), 1,2-dimyristoyl-sn-phosphatidylcholine (DMPC), 1,2-dihexadecanoyl-sn-glycero-3-phosphatidylcholine (DPPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1-oleoyl-2-hydroxy-sn-glycero-3-phosphoethanolamine, 1-stearoyl-2-hydroxy-sn-glycero-3-phosphoethanolamine, 1,2-distearoyl-sn-glycero-3-phosphoethanolamine, and 1,2-dielaidoyl-sn-glycero-3-phosphoethanolamine, 1,2-dioleoyl-sn-glycero-3-phosphoethanolamine-N-[azido(polyethylene glycol)] (DOPE-PEG-azide), 1,2-dioleoyl-sn-glycero-3-phosphoethanolamine-N-[maleimide(polyethylene glycol)] (DOPE-PEG-maleimide), 1,2-dipalmitoyl-sn-glycero-3-phosphoethanolamine-N-[azido(polyethylene glycol)] (DPPE-PEG-azide), 1,2-dipalmitoyl-sn-glycero-3-phosphoethanolamine-N-[maleimide(polyethylene glycol)] (DPPE-PEG-maleimide),1,2-distearoyl-sn-glycero-3-phosphoethanolamine-N-[azido(polyethylene glycol)] (DSPE-PEG-azide), and 1,2-distearoyl-sn-glycero-3-phosphoethanolamine-N-[maleimide(polyethylene glycol)] (DSPE-PEG-maleimide), 1,2-dihexadecanoyl-sn-glycero-3-phosphoethanolamine (DPPE), or a combination thereof.

14. The nanoparticle of claim 11, wherein the plurality of lipid groups comprises 1,2-dipalmitoyl-sn-glycero-3-phosphocholine (DPPC).

15. The nanoparticle of claim 11, wherein the plurality of lipid groups comprises 1,2-dioleoyl-sn-glycero-3-phosphocholine (DOPC).

16. The nanoparticle of any one of claims 1-15, wherein diameter of the nanoparticle is about 20 nanometers (nm) to about 150 nm.

17. The nanoparticle of any one of claims 1-15, wherein diameter of the nanoparticle is less than or equal to about 100 nanometers.

18. The nanoparticle of any one of claims 1-15, wherein diameter of the nanoparticle is less than or equal to about 80 nanometers.

19. The nanoparticle of any one of claims 1-18, wherein the nanoparticle comprises about 10 to about 200 oligonucleotides.

20. The nanoparticle of claims 19, wherein the nanoparticle comprises 75 oligonucleotides.

21. The nanoparticle of any one of claims 1-20, wherein the ratio of oligonucleotide to tumor cell lysate is about 0.5 nmol to about 25 nmol: about 5 μg to about 150 μg.

22. The nanoparticle of any one of claims 1-20, wherein the ratio of oligonucleotide to tumor cell lysate is about 5 nmol oligonucleotide:20 μg tumor cell lysate.

23. The nanoparticle of any one of claims 1-22, wherein the oxidized tumor cell lysate is derived from a tumor cell exposed to hypochlorous acid (HOCl), hydrogen peroxide, sodium hypochlorite, sodium chlorite, nitric acid, or sulfur.

24. The nanoparticle of claim 23, wherein the tumor cell is exposed to about 10 μM to about 100 μM HOCl.

25. The nanoparticle of claim 23, wherein the tumor cell is exposed to about 60 μM HOCl.

26. The nanoparticle of any one of claims 1-25, wherein the tumor cell lysate is derived from a breast cancer cell, peritoneum cancer cell, cervical cancer cell, colon cancer cell, rectal cancer cell, esophageal cancer cell, eye cancer cell, liver cancer cell, pancreatic cancer cell, larynx cancer cell, lung cancer cell, skin cancer cell, ovarian cancer cell, prostate cancer cell, stomach cancer cell, testicular cancer cell, thyroid cancer cell, brain cancer cell, or a combination thereof.

27. The nanoparticle of any one of claims 1-25, wherein the tumor cell lysate is derived from a triple negative breast cancer (TNBC) cell.

28. The nanoparticle of any one of claims 1-27, wherein the oligonucleotide is DNA.

29. The nanoparticle of any one of claims 1-27, wherein the oligonucleotide is RNA.

30. A pharmaceutical formulation comprising the nanoparticle of any one of claims 1-29 and a pharmaceutically acceptable carrier or diluent.

31. A method of making a liposomal nanoparticle comprising:
exposing a tumor cell to an oxidizing agent to produce an oxidized tumor cell; then
isolating lysate from the oxidized tumor cell; then
contacting a lipid film with the lysate to produce a small unilamellar vesicle (SUV) comprising the lysate encapsulated therein; then
adding an oligonucleotide to the SUV to make the liposomal nanoparticle.

32. The method of claim 31, wherein the oxidizing agent is hypochlorous acid (HOCl).

33. The method of claim 31 or claim 32, wherein the tumor cell is exposed to about 10 μM to about 100 μM of the oxidizing agent.

34. The method of any one of claims 31-33, wherein the tumor cell is exposed to about 60 μM HOCl.

35. The method of any one of claims 31-34, wherein the oligonucleotide is a Toll-Like Receptor (TLR) agonist.

36. The method of claim 35, wherein the TLR agonist is a toll-like receptor 1 (TLR1) agonist, a toll-like receptor 2 (TLR2) agonist, a toll-like receptor 3 (TLR3) agonist, a toll-like receptor 4 (TLR4) agonist, a toll-like receptor 5 (TLR5) agonist, a toll-like receptor 6 (TLR6) agonist, a toll-like receptor 7 (TLR7) agonist, a toll-like receptor 8 (TLR8) agonist, a toll-like receptor 9 (TLR9) agonist, a toll-like receptor 10 (TLR10) agonist, a toll-like receptor 11 (TLR11) agonist, a toll-like receptor 12 (TLR12) agonist, a toll-like receptor 13 (TLR13) agonist, or a combination thereof.

37. The method of claim 35 or claim 36, wherein the TLR agonist is a toll-like receptor 3 (TLR3) agonist, a toll-like receptor 7 (TLR7) agonist, a toll-like receptor 8 (TLR8) agonist, a toll-like receptor 9 (TLR9) agonist, or a combination thereof.

38. The method of any one of claims 31-37, wherein the liposomal nanoparticle is about 50 nanometers (nm) to about 100 nanometers (nm) in diameter.

39. The method of any one of claims 31-37, wherein the liposomal nanoparticle is less than about 100 nm in diameter.

40. The method of any one of claims 31-37, wherein the liposomal nanoparticle is about 80 nm in diameter.

41. The method of any one of claims 31-40, wherein the oligonucleotide is an oligonucleotide-lipid conjugate containing a lipophilic tethered group, wherein said lipophilic tethered group is adsorbed into the surface of the SUV.

42. The method of claim 41, wherein the lipophilic tethered group comprises tocopherol or cholesterol.

43. The method of claim 42, wherein tocopherol is a tocopherol derivative, alpha-tocopherol, beta-tocopherol, gamma-tocopherol, or delta-tocopherol.

44. The method of any one of claims 31-43, wherein the oligonucleotide comprises RNA or DNA.

45. The method of claim 44, wherein the oligonucleotide is DNA.

46. The method of any one of claims 31-45, wherein the oligonucleotide is a modified oligonucleotide.

47. The method of any one of claims 31-46, wherein the ratio of oligonucleotide to tumor cell lysate is about 0.5 nmol to about 25 nmol: about 5 μg to about 150 μg.

48. The method of any one of claims 31-47, wherein the ratio of oligonucleotide to tumor cell lysate is about 5 nmol oligonucleotide:20 μg tumor cell lysate.

49. An antigenic composition comprising the nanoparticle of any one of claims 1-29 in a pharmaceutically acceptable carrier, diluent, stabilizer, preservative, or adjuvant, or the pharmaceutical formulation of claim 30, wherein the antigenic composition is capable of generating an immune response including antibody generation or a protective immune response in a mammalian subject.

50. The antigenic composition of claim 49, wherein the antibody response is a neutralizing antibody response or a protective antibody response.

51. A method of producing an immune response to cancer in a subject, comprising administering to the subject an effective amount of the antigenic composition of claim 49 or claim 50, thereby producing an immune response to cancer in the subject.

52. The method of claim 51, wherein the cancer is breast cancer, peritoneum cancer, cervical cancer, colon cancer, rectal cancer, esophageal cancer, eye cancer, liver cancer, pancreatic cancer, larynx cancer, lung cancer, skin cancer, ovarian cancer, prostate cancer, stomach cancer, testicular cancer, thyroid cancer, brain cancer, or a combination thereof.

53. The method of claim 51, wherein the cancer is breast cancer.

54. The method of claim 53, wherein the breast cancer is triple negative breast cancer (TNBC).

55. A method of treating cancer in a subject in need thereof, comprising administering to the subject an effective amount of the nanoparticle of any one of claims 1-29, the pharmaceutical formulation of claim 30, or the antigenic composition of claim 49 or claim 50, thereby treating cancer in the subject.

56. The method of claim 55, wherein the administering is subcutaneous.

57. The method of claim 55, wherein the administering is intravenous, intraperitoneal, intranasal, or intramuscular.


SEQUENCE LISTING (3 sequences)

SEQ ID NO: 1 - DNA, 20 bp, Artificial Sequence
  Synthetic Primer
tccatgacgttcctgacgtt

SEQ ID NO: 2 - DNA, 20 bp, Artificial Sequence
  Synthetic Primer
  misc_feature (20)..(20): Spacer-18 (hexaethyleneglycol))2 Chol
tccatgacgttcctgacgtt

SEQ ID NO: 3 - DNA, 20 bp, Artificial Sequence
  Synthetic Primer
  misc_feature (20)..(20): Cy5-(Spacer-18 (hexaethyleneglycol))2 Chol
tccatgacgttcctgacgtt